SEQUENCE LISTING (200 sequences)

SEQ ID NO: 1 - DNA, 30 bp, Artificial
  Probe
cggyatwtcngatgcnatygtyaayaaytt

SEQ ID NO: 2 - DNA, 30 bp, Artificial
  Probe
cggyatwtcngatgtnatygtyaayaaytt

SEQ ID NO: 3 - DNA, 31 bp, Artificial
  Probe
yggyatytcwgayatmattgtwaayaayttc

SEQ ID NO: 4 - DNA, 30 bp, Artificial
  Probe
gggcatctcygaygcaatygtyaataaytt

SEQ ID NO: 5 - DNA, 30 bp, Artificial
  Probe
gggtatctcagatgccattgttaataattt

SEQ ID NO: 6 - DNA, 30 bp, Artificial
  Probe
gggyatytcwgaygcyattgttaataactt

SEQ ID NO: 7 - DNA, 20 bp, Artificial
  Primer
accactattattgccacrgc

SEQ ID NO: 8 - DNA, 20 bp, Artificial
  Primer
actaccatyattgccacggc

SEQ ID NO: 9 - DNA, 39 bp, Artificial
  Primer
cacttctcagtgtggcgcgtcggggccttatcagtcgtc

SEQ ID NO: 10 - DNA, 39 bp, Artificial
  Primer
cacttctcagtgtggcgcgtcgwggcctcatcagtcgtc

SEQ ID NO: 11 - DNA, 40 bp, Artificial
  Primer
caggcctgcttcgcgaggtatttcgccaccagcaggaaaa

SEQ ID NO: 12 - DNA, 39 bp, Artificial
  Primer
caggcctgcttcgtgaggtacctcgccaccagcaggaaa

SEQ ID NO: 13 - DNA, 19 bp, Artificial
  Primer
cggggaataacwgatgggc

SEQ ID NO: 14 - DNA, 19 bp, Artificial
  Primer
cgagggataacagatgggc

SEQ ID NO: 15 - DNA, 20 bp, Artificial
  Primer
gcaacaatggcatgagcycg

SEQ ID NO: 16 - DNA, 20 bp, Artificial
  Primer
taattgcaactgcagatgcc

SEQ ID NO: 17 - DNA, 41 bp, Artificial
  Primer
catttttcagtatgcctagtgcgtggcctcatacagtcctc

SEQ ID NO: 18 - DNA, 37 bp, Artificial
  Primer
tcccggcctgttgcgtgactcgccacccgaaaggaag

SEQ ID NO: 19 - DNA, 20 bp, Artificial
  Primer
cgcggaatgaccgatggtct

SEQ ID NO: 20 - DNA, 20 bp, Artificial
  Primer
gcaactatagcgtgagcccg

SEQ ID NO: 21 - DNA, 20 bp, Artificial
  Primer
accacaatyatagccacggc

SEQ ID NO: 22 - DNA, 20 bp, Artificial
  Primer
accacaattatagctacggc

SEQ ID NO: 23 - DNA, 20 bp, Artificial
  Primer
accacaatcatagcaacggc

SEQ ID NO: 24 - DNA, 40 bp, Artificial
  Primer
cacttctcagtgtggcgrgtggggccttattcagtccatc

SEQ ID NO: 25 - DNA, 40 bp, Artificial
  Primer
cacttctctgtgtgrcgggtaggggccttatccagtcatc

SEQ ID NO: 26 - DNA, 40 bp, Artificial
  Primer
cacctctctgtgtggcgggtaggggtctcatccagtcatc

SEQ ID NO: 27 - DNA, 40 bp, Artificial
  Primer
cacttctctgtgtggcgggtagaggccttatccagtaatc

SEQ ID NO: 28 - DNA, 38 bp, Artificial
  Primer
ccggcctgcttcgtgaggcctcgccaccagcaaggaaa

SEQ ID NO: 29 - DNA, 38 bp, Artificial
  Primer
cggcctgcgcgygaggtccctctccaccagcaaggaaa

SEQ ID NO: 30 - DNA, 38 bp, Artificial
  Primer
tggtttgctgcgcgaggtcctccccgccagcaaggaaa

SEQ ID NO: 31 - DNA, 38 bp, Artificial
  Primer
tggtctgctgcgtgaggtcctccccaccagcgaggaaa

SEQ ID NO: 32 - DNA, 39 bp, Artificial
  Primer
cctggtttgttacgtgaggtgctccccaccagcgaggaa

SEQ ID NO: 33 - DNA, 40 bp, Artificial
  Primer
ccggtttattgcgtgaggtgcctctccaccagcaaggaaa

SEQ ID NO: 34 - DNA, 20 bp, Artificial
  Primer
cgaggtataacagargggcg

SEQ ID NO: 35 - DNA, 20 bp, Artificial
  Primer
cggggtatcacagaggggcg

SEQ ID NO: 36 - DNA, 20 bp, Artificial
  Primer
gcgactatagcgtgagcycg

SEQ ID NO: 37 - DNA, 20 bp, Artificial
  Primer
gcaactatagcatgagctcg

SEQ ID NO: 38 - DNA, 20 bp, Artificial
  Primer
actacrattattgccacggc

SEQ ID NO: 39 - DNA, 20 bp, Artificial
  Primer
actactattattgcwacggc

SEQ ID NO: 40 - DNA, 39 bp, Artificial
  Primer
cacttctccgtgtggcgggtgtgggctgatccagtcatc

SEQ ID NO: 41 - DNA, 39 bp, Artificial
  Primer
cacttctccgtgtgacgagtgggggctgatycagtcctc

SEQ ID NO: 42 - DNA, 39 bp, Artificial
  Primer
catttctccgtatgacgggtgggggctgattcagtcatc

SEQ ID NO: 43 - DNA, 39 bp, Artificial
  Primer
catttctctgtctggcgagtgggggctgattcagtcctc

SEQ ID NO: 44 - DNA, 40 bp, Artificial
  Primer
ggggctcctccgtgaggtgatctgaccaccagaaaggaaa

SEQ ID NO: 45 - DNA, 40 bp, Artificial
  Primer
ggggcttcttcgtgaggtgatctcgccaccagaaaggaag

SEQ ID NO: 46 - DNA, 40 bp, Artificial
  Primer
gggactccttcgtgaggtcatctggccaccagaaaggaaa

SEQ ID NO: 47 - DNA, 41 bp, Artificial
  Primer
cggggcttcttcgtgaggttacctggccgccagaaaggaaa

SEQ ID NO: 48 - DNA, 20 bp, Artificial
  Primer
cgccgtatgactgatggacg

SEQ ID NO: 49 - DNA, 19 bp, Artificial
  Primer
ggggtacaactgacgggcg

SEQ ID NO: 50 - DNA, 20 bp, Artificial
  Primer
cgcggtatgactgatggacg

SEQ ID NO: 51 - DNA, 20 bp, Artificial
  Primer
cgaggtatgactgatgggcg

SEQ ID NO: 52 - DNA, 19 bp, Artificial
  Primer
gccacgattgcgtgggctc

SEQ ID NO: 53 - DNA, 19 bp, Artificial
  Primer
gccacaatggcatgagccc

SEQ ID NO: 54 - DNA, 19 bp, Artificial
  Primer
gccacaatagcgtgrgccc

SEQ ID NO: 55 - DNA, 20 bp, Artificial
  Primer
acgcgccacactgagaagtg

SEQ ID NO: 56 - DNA, 40 bp, Artificial
  Primer
atttcgccaccagcaggaaaaacctcgcgaagcaggcctg

SEQ ID NO: 57 - DNA, 40 bp, Artificial
  Primer
acctcgccaccagcaggaagaacctcgcgaagcaggcctg

SEQ ID NO: 58 - DNA, 38 bp, Artificial
  Primer
gcccatcwgttattccccgtggcaagacggcgggaagg

SEQ ID NO: 59 - DNA, 38 bp, Artificial
  Primer
gcccatctgttatccctcgtggcaagacggcgggaagg

SEQ ID NO: 60 - DNA, 20 bp, Artificial
  Primer
agccaactgatggaaggcrc

SEQ ID NO: 61 - DNA, 20 bp, Artificial
  Primer
cctgacgccaatgttgacac

SEQ ID NO: 62 - DNA, 20 bp, Artificial
  Primer
actaggcatactgaaaaatg

SEQ ID NO: 63 - DNA, 38 bp, Artificial
  Primer
ctcgccacccgaaaggaagcggcctgttgcgtgaggtg

SEQ ID NO: 64 - DNA, 39 bp, Artificial
  Primer
gacccatcggtcattccgccatgatgaaggtggaaacgc

SEQ ID NO: 65 - DNA, 19 bp, Artificial
  Primer
agcaagctgatggaaggcg

SEQ ID NO: 66 - DNA, 20 bp, Artificial
  Primer
cctgaccgcaatgttgacgt

SEQ ID NO: 67 - DNA, 20 bp, Artificial
  Primer
acycgccacactgagaagtg

SEQ ID NO: 68 - DNA, 20 bp, Artificial
  Primer
acccgycacacagagaagtg

SEQ ID NO: 69 - DNA, 20 bp, Artificial
  Primer
acccgccacacagagaggtg

SEQ ID NO: 70 - DNA, 20 bp, Artificial
  Primer
acccgccacacagagaagtg

SEQ ID NO: 71 - DNA, 38 bp, Artificial
  Primer
cctcgccaccagcaaggaaaccggcctgcttcgtgagg

SEQ ID NO: 72 - DNA, 38 bp, Artificial
  Primer
cctctccaccagcaaggaaaggcctgctgcgygaggtc

SEQ ID NO: 73 - DNA, 38 bp, Artificial
  Primer
cctccccgccagcaaggaaatggtttgctgcgcgaggt

SEQ ID NO: 74 - DNA, 38 bp, Artificial
  Primer
cctccccaccagcgaggaaatggtctgctgcgtgaggt

SEQ ID NO: 75 - DNA, 39 bp, Artificial
  Primer
gctccccaccagcgaggaacctggtttgttacgtgaggt

SEQ ID NO: 76 - DNA, 39 bp, Artificial
  Primer
cctctccaccagcaaggaaaccggtttattgcgtgaggt

SEQ ID NO: 77 - DNA, 38 bp, Artificial
  Primer
cgcccttctgtgatacctcgacaggacggcgggaaggc

SEQ ID NO: 78 - DNA, 37 bp, Artificial
  Primer
gcccctccgtgatacctcggcaagacggcgggaatgc

SEQ ID NO: 79 - DNA, 38 bp, Artificial
  Primer
gcccctctgtgataccccggrcaagatggcgggaaggc

SEQ ID NO: 80 - DNA, 37 bp, Artificial
  Primer
gcccctctgtgataccccgacaagacggcgggaaggc

SEQ ID NO: 81 - DNA, 20 bp, Artificial
  Primer
agccarctgatggtaagcac

SEQ ID NO: 82 - DNA, 20 bp, Artificial
  Primer
agccaactgrtggtaagcac

SEQ ID NO: 83 - DNA, 20 bp, Artificial
  Primer
aaccctgatcagaacctcgg

SEQ ID NO: 84 - DNA, 21 bp, Artificial
  Primer
aaccctgaccagaaccttgag

SEQ ID NO: 85 - DNA, 20 bp, Artificial
  Primer
acccgccacacggagaagtg

SEQ ID NO: 86 - DNA, 20 bp, Artificial
  Primer
actcgtcacacggagaagtg

SEQ ID NO: 87 - DNA, 20 bp, Artificial
  Primer
acccgtcatacggagaaatg

SEQ ID NO: 88 - DNA, 20 bp, Artificial
  Primer
actcgccagacagagaaatg

SEQ ID NO: 89 - DNA, 40 bp, Artificial
  Primer
tctgaccaccagaaaggaaaaggggctcctccgtgaggtg

SEQ ID NO: 90 - DNA, 40 bp, Artificial
  Primer
tctcgccaccagaaaggaagaggggcttcttcgtgaggtg

SEQ ID NO: 91 - DNA, 40 bp, Artificial
  Primer
tctggccaccagaaaggaaaagggactccttcgtgaggtc

SEQ ID NO: 92 - DNA, 41 bp, Artificial
  Primer
cctggccgccagaaaggaaaacggggcttcttcgtgaggtt

SEQ ID NO: 93 - DNA, 38 bp, Artificial
  Primer
cgtccatcagtcataccgcgggcaagagggcgggaagg

SEQ ID NO: 94 - DNA, 38 bp, Artificial
  Primer
cgtccgtcagttataccccgggcaggaaggggggaatg

SEQ ID NO: 95 - DNA, 38 bp, Artificial
  Primer
cgtccatcagtcataccgcgggcaagagggtgggaatg

SEQ ID NO: 96 - DNA, 41 bp, Artificial
  Primer
cgtccatcagtcataccgcgttgacaagaaggtggaaatgc

SEQ ID NO: 97 - DNA, 19 bp, Artificial
  Primer
gccagctggtggttagcac

SEQ ID NO: 98 - DNA, 19 bp, Artificial
  Primer
gccagctgatggtacgcgc

SEQ ID NO: 99 - DNA, 19 bp, Artificial
  Primer
gcaagctggtggtaagcgc

SEQ ID NO: 100 - DNA, 19 bp, Artificial
  Primer
cgaagctggtgataggcgc

SEQ ID NO: 101 - DNA, 20 bp, Artificial
  Primer
acaatgttgacacacttgat

SEQ ID NO: 102 - DNA, 20 bp, Artificial
  Primer
acaatgttgatacgcttgat

SEQ ID NO: 103 - DNA, 20 bp, Artificial
  Primer
gcaatgttgccacgcttgat

SEQ ID NO: 104 - DNA, 30 bp, Artificial
  Probe
cggcatatcggatgccattgtcaacaactt

SEQ ID NO: 105 - DNA, 30 bp, Artificial
  Probe
cggcatttcggatgtkattgtcaayaactt

SEQ ID NO: 106 - DNA, 30 bp, Artificial
  Probe
cggtatttcrgatgtgattgtyaayaactt

SEQ ID NO: 107 - DNA, 30 bp, Artificial
  Probe
cggtatatcagatgtgattgttaacaattt

SEQ ID NO: 108 - DNA, 30 bp, Artificial
  Probe
cggcatatctgatgtgatcgttaacaactt

SEQ ID NO: 109 - DNA, 30 bp, Artificial
  Probe
cggyatatcagatgtrattgtcaayaactt

SEQ ID NO: 110 - DNA, 30 bp, Artificial
  Probe
cggcatatcagatgtgattgtcaataactt

SEQ ID NO: 111 - DNA, 30 bp, Artificial
  Probe
cggcatatcmgatgtgattgtcaataactt

SEQ ID NO: 112 - DNA, 30 bp, Artificial
  Probe
cggcatatcagatgtgattgttaataattt

SEQ ID NO: 113 - DNA, 30 bp, Artificial
  Probe
cggcatatcggatgtcattgtcaacaactt

SEQ ID NO: 114 - DNA, 30 bp, Artificial
  Probe
cggyatttcagatatcattgtaaataattt

SEQ ID NO: 115 - DNA, 30 bp, Artificial
  Probe
cggcatttctgacataattgttaataactt

SEQ ID NO: 116 - DNA, 30 bp, Artificial
  Probe
gggcatctccgatgcaatcgttaataactt

SEQ ID NO: 117 - DNA, 30 bp, Artificial
  Probe
gggcatctctgacgcaatcgtyaataaytt

SEQ ID NO: 118 - DNA, 30 bp, Artificial
  Probe
cggcatttctgatgctattgtaaataactt

SEQ ID NO: 119 - DNA, 30 bp, Artificial
  Probe
tgcgatctccgatgcaatcgttaataactt

SEQ ID NO: 120 - DNA, 30 bp, Artificial
  Probe
gggcatttccgatgcaatcgttaataactt

SEQ ID NO: 121 - DNA, 30 bp, Artificial; identical to SEQ ID NO: 5
  Probe

SEQ ID NO: 122 - DNA, 30 bp, Artificial
  Probe
cggtatctctgacgccattgttaataactt

SEQ ID NO: 123 - DNA, 30 bp, Artificial
  Probe
cggtatttctgacgccattgtcaataactt

SEQ ID NO: 124 - DNA, 30 bp, Artificial
  Probe
tggcatttcagatgccattgttaataactt

SEQ ID NO: 125 - DNA, 30 bp, Artificial
  Probe
tggtatttctgatgctattgttaataactt

SEQ ID NO: 126 - DNA, 30 bp, Artificial
  Probe
cggcatctccgatgccatagtgaataattt

SEQ ID NO: 127 - DNA, 30 bp, Artificial
  Probe
tggtatctctgacgccatagtcaacaattt

SEQ ID NO: 128 - DNA, 27 bp, Artificial
  Probe
ggcatatcggatgccattgtcaacaac

SEQ ID NO: 129 - DNA, 27 bp, Artificial
  Probe
ggcatttcggatgtkattgtcaayaac

SEQ ID NO: 130 - DNA, 27 bp, Artificial
  Probe
ggtatttcrgatgtgattgtyaayaac

SEQ ID NO: 131 - DNA, 27 bp, Artificial
  Probe
ggtatatcagatgtgattgttaacaat

SEQ ID NO: 132 - DNA, 27 bp, Artificial
  Probe
ggcatatctgatgtgatcgttaacaac

SEQ ID NO: 133 - DNA, 27 bp, Artificial
  Probe
ggyatatcagatgtrattgtcaayaac

SEQ ID NO: 134 - DNA, 27 bp, Artificial
  Probe
ggcatatcagatgtgattgtcaataac

SEQ ID NO: 135 - DNA, 27 bp, Artificial
  Probe
ggcatatcmgatgtgattgtcaataac

SEQ ID NO: 136 - DNA, 27 bp, Artificial
  Probe
ggcatatcagatgtgattgttaataat

SEQ ID NO: 137 - DNA, 27 bp, Artificial
  Probe
ggcatatcggatgtcattgtcaacaac

SEQ ID NO: 138 - DNA, 27 bp, Artificial
  Probe
ggyatttcagatatcattgtaaataat

SEQ ID NO: 139 - DNA, 27 bp, Artificial
  Probe
ggcatttctgacataattgttaataac

SEQ ID NO: 140 - DNA, 27 bp, Artificial
  Probe
ggcatctccgatgcaatcgttaataac

SEQ ID NO: 141 - DNA, 27 bp, Artificial
  Probe
ggcatctctgacgcaatcgtyaataay

SEQ ID NO: 142 - DNA, 27 bp, Artificial
  Probe
ggcatttctgatgctattgtaaataac

SEQ ID NO: 143 - DNA, 27 bp, Artificial
  Probe
gcgatctccgatgcaatcgttaataac

SEQ ID NO: 144 - DNA, 27 bp, Artificial
  Probe
ggcatttccgatgcaatcgttaataac

SEQ ID NO: 145 - DNA, 27 bp, Artificial
  Probe
ggtatctcagatgccattgttaataat

SEQ ID NO: 146 - DNA, 27 bp, Artificial
  Probe
ggtatctctgacgccattgttaataac

SEQ ID NO: 147 - DNA, 27 bp, Artificial
  Probe
ggtatttctgacgccattgtcaataac

SEQ ID NO: 148 - DNA, 27 bp, Artificial
  Probe
ggcatttcagatgccattgttaataac

SEQ ID NO: 149 - DNA, 27 bp, Artificial
  Probe
ggtatttctgatgctattgttaataac

SEQ ID NO: 150 - DNA, 27 bp, Artificial
  Probe
ggcatctccgatgccatagtgaataat

SEQ ID NO: 151 - DNA, 27 bp, Artificial
  Probe
ggtatctctgacgccatagtcaacaat

SEQ ID NO: 152 - DNA, 23 bp, Artificial
  Probe
catatcggatgccattgtcaaca

SEQ ID NO: 153 - DNA, 23 bp, Artificial
  Probe
catttcggatgtkattgtcaaya

SEQ ID NO: 154 - DNA, 23 bp, Artificial
  Probe
tatttcrgatgtgattgtyaaya

SEQ ID NO: 155 - DNA, 23 bp, Artificial
  Probe
tatatcagatgtgattgttaaca

SEQ ID NO: 156 - DNA, 23 bp, Artificial
  Probe
catatctgatgtgatcgttaaca

SEQ ID NO: 157 - DNA, 23 bp, Artificial
  Probe
yatatcagatgtrattgtcaaya

SEQ ID NO: 158 - DNA, 23 bp, Artificial
  Probe
catatcagatgtgattgtcaata

SEQ ID NO: 159 - DNA, 23 bp, Artificial
  Probe
catatcmgatgtgattgtcaata

SEQ ID NO: 160 - DNA, 23 bp, Artificial
  Probe
catatcagatgtgattgttaata

SEQ ID NO: 161 - DNA, 23 bp, Artificial
  Probe
catatcggatgtcattgtcaaca

SEQ ID NO: 162 - DNA, 23 bp, Artificial
  Probe
yatttcagatatcattgtaaata

SEQ ID NO: 163 - DNA, 23 bp, Artificial
  Probe
catttctgacataattgttaata

SEQ ID NO: 164 - DNA, 23 bp, Artificial
  Probe
catctccgatgcaatcgttaata

SEQ ID NO: 165 - DNA, 23 bp, Artificial
  Probe
catctctgacgcaatcgtyaata

SEQ ID NO: 166 - DNA, 23 bp, Artificial
  Probe
catttctgatgctattgtaaata

SEQ ID NO: 167 - DNA, 23 bp, Artificial
  Probe
gatctccgatgcaatcgttaata

SEQ ID NO: 168 - DNA, 23 bp, Artificial
  Probe
catttccgatgcaatcgttaata

SEQ ID NO: 169 - DNA, 23 bp, Artificial
  Probe
tatctcagatgccattgttaata

SEQ ID NO: 170 - DNA, 23 bp, Artificial
  Probe
tatctctgacgccattgttaata

SEQ ID NO: 171 - DNA, 23 bp, Artificial
  Probe
tatttctgacgccattgtcaata

SEQ ID NO: 172 - DNA, 23 bp, Artificial
  Probe
catttcagatgccattgttaata

SEQ ID NO: 173 - DNA, 23 bp, Artificial
  Probe
tatttctgatgctattgttaata

SEQ ID NO: 174 - DNA, 23 bp, Artificial
  Probe
catctccgatgccatagtgaata

SEQ ID NO: 175 - DNA, 23 bp, Artificial
  Probe
tatctctgacgccatagtcaaca

SEQ ID NO: 176 - DNA, 20 bp, Artificial
  Probe
tatcggatgccattgtcaac

SEQ ID NO: 177 - DNA, 20 bp, Artificial
  Probe
tttcggatgtkattgtcaay

SEQ ID NO: 178 - DNA, 20 bp, Artificial
  Probe
tttcrgatgtgattgtyaay

SEQ ID NO: 179 - DNA, 20 bp, Artificial
  Probe
tatcagatgtgattgttaac

SEQ ID NO: 180 - DNA, 20 bp, Artificial
  Probe
tatctgatgtgatcgttaac

SEQ ID NO: 181 - DNA, 20 bp, Artificial
  Probe
tatcagatgtrattgtcaay

SEQ ID NO: 182 - DNA, 20 bp, Artificial
  Probe
tatcagatgtgattgtcaat

SEQ ID NO: 183 - DNA, 20 bp, Artificial
  Probe
tatcmgatgtgattgtcaat

SEQ ID NO: 184 - DNA, 20 bp, Artificial
  Probe
tatcagatgtgattgttaat

SEQ ID NO: 185 - DNA, 20 bp, Artificial
  Probe
tatcggatgtcattgtcaac

SEQ ID NO: 186 - DNA, 20 bp, Artificial
  Probe
tttcagatatcattgtaaat

SEQ ID NO: 187 - DNA, 20 bp, Artificial
  Probe
tttctgacataattgttaat

SEQ ID NO: 188 - DNA, 20 bp, Artificial
  Probe
tctccgacgcaatcgtcaat

SEQ ID NO: 189 - DNA, 20 bp, Artificial
  Probe
tctctgacgcaatcgttaat

SEQ ID NO: 190 - DNA, 20 bp, Artificial
  Probe
tttctgatgctattgtaaat

SEQ ID NO: 191 - DNA, 20 bp, Artificial
  Probe
tctccgatgcaatcgttaat

SEQ ID NO: 192 - DNA, 20 bp, Artificial
  Probe
tttccgatgcaatcgttaat

SEQ ID NO: 193 - DNA, 20 bp, Artificial
  Probe
tctcagatgccattgttaat

SEQ ID NO: 194 - DNA, 20 bp, Artificial
  Probe
tctctgacgccattgttaat

SEQ ID NO: 195 - DNA, 20 bp, Artificial
  Probe
tttctgacgccattgtcaat

SEQ ID NO: 196 - DNA, 20 bp, Artificial
  Probe
tttcagatgccattgttaat

SEQ ID NO: 197 - DNA, 20 bp, Artificial
  Probe
tttctgatgctattgttaat

SEQ ID NO: 198 - DNA, 20 bp, Artificial
  Probe
tctccgatgccatagtgaat

SEQ ID NO: 199 - DNA, 20 bp, Artificial
  Probe
tctctgacgccatagtcaac

SEQ ID NO: 200 - protein, 127 aa, Hepatitis E virus US1
ADVCELIRGAYPKIQTTSRVLRSLFWNEPAIGQKLVFTQAAKAANPGAITVHEAQGATFTETTIIATADARGLIQSSRAHAIVALTRHTEKCVILDAPGLLREVGISDVIVNNFFLAGGEVGHHRPS

(19) United States
(12) Patent Application Publication
(10) Pub. No.: US 2010/0173283 A1
(43) Pub. Date: Jul. 8, 2010

(54) METHOD OF PREDICTING POTENTIAL SEVERITY OF HEPATITIS E, PROBE SETS, AND PRIMER SETS

(76) Inventors: Kazuaki Takahashi, Machida-shi (JP); Natsumi Abe, Kawasaki-shi (JP); Sheikh Mohammad Fazle Akbar, Tokyo (JP); Michie Hashimoto, Tokyo (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/642,553

(22) Filed: Dec. 18, 2009

(30) Foreign Application Priority Data
Dec. 19, 2008 (JP) 2008-324630

(51) Int. Cl. C12Q 1/70

(52) U.S. Cl. 435/5

(57) ABSTRACT

The present invention provides a method of predicting potential severity of hepatitis, includes determining that hepatitis in a subject is potentially severe when it is detected that any amino acid is mutated to an amino acid of genotype-4, the any amino acid being amino acid of an amino acid sequence in a region encoded by ORF1 of an HEV genome RNA of genotype 3, and the HEV genome RNA being contained in a specimen nucleic acid taken from the subject infected with genotype-3 HEV.

### CROSS-REFERENCE TO RELATED APPLICATIONS

This application is based upon and claims the benefit of priority from prior Japanese Patent Application No. 2008-324630, filed Dec. 19, 2008, the entire contents of which are incorporated herein by reference.

### BACKGROUND OF THE INVENTION

1. Field of the Invention

The present invention relates to the prediction of the severity of hepatitis E.

2. Description of the Related Art

When hepatitis E is detected in advanced countries such as Japan, the United States, and Europe, it is understood to be an imported infectious disease in most cases, because oral infection occurring in an insanitary environment is considered to be the main cause. However, it has been found that an indigenous strain of hepatitis E virus (HEV) exists in Japan and is caused by eating the meat and organs of pigs or wild animals. The number of reported cases of HIV infection in Japan has been increasing since 2000 (Hiroaki Okamoto, Present Situation of Hepatitis E in Japan, Liver, 2006; 47(3):379-383). Severe cases have been reported and thus there is an urgent need to define the route of infection and establish preventive treatment.

In an emergency research project to overcome hepatitis carried out by the Ministry of Health, Labour and Welfare, a study group (the MHLW Study Group) conducting research into the infection route, host range, genetic diversity, infection prevention, diagnosis, and treatment of hepatitis E was set up in 2006. This study group has analyzed 254 cases of HIV infection collected from around Japan in order to resolve the entity of hepatitis E in Japan (Toshinori Abe, Tatsuya Aikawa, Masahiro Akabane, et al., Statistical, Epidemiological, and Virological Characteristics of Hepatitis E Virus Infection in Japan: Analysis based on 254 cases [national statistics], Liver, 2006; 47(8):384-391).

As a result, 188 (77%) out of 243 subjects who developed hepatitis E were males and 55 (23%) were females. Out of 242 subjects, 105 (43%) were between the ages of 40 and 59, 74 (31%) were 60 years of age or older, and 63 (26%) were younger than 40. Considering the results on a regional basis, there are 123 (54%) subjects in Hokkaido, 48 (21%) subjects in the Kanto and Koshinetsu districts, and 18 (8%) subjects in the Tohoku district.

In the analysis, the detection frequency according to each HEV genotype has been reported. The most frequently detected genotype in Japan is genotype 3 (135 subjects, 61%), the second most frequently detected genotype is genotype 4 (78 subjects, 36%), and the least frequently detected genotype is genotype 1 (7 subjects, 3%). Genotype 2 has not been detected. While the top genotype domestically is genotype 3, many instances of genotype 4 have been significantly detected in HEV positive subjects who developed sever hepatitis such as the severe form of acute hepatitis and fulminant hepatitis. In the above-described analysis, seven (12%) out of 59 cases of inapparent infection are genotype 4. On the other hand, 48 (37%) out of 130 cases of acute hepatitis are genotype 4, and 23 (74%) out of 31 cases of acute hepatitis severe form and fulminant hepatitis are genotype 4. This indicates that the frequency of genotype 4 is increased with the deterioration of hepatitis.

Therefore, the process of identifying what genotype of strain the HEV positive subject is infected with provides important information for predicting the subject's pathological condition and performing appropriate treatment.

When HEVs obtained from the body of pigs and wild animals which lead to hepatitis are analyzed, the same strain is detected from subjects who became HEV positive in a habitat for the animals in many cases. That is, when pigs and wild animals with genotype 4 which is a severe strain are removed from distribution so that humans do not ingest and measures to vaccinate against the genotype-4 strain are strengthened, humans may be protected from severe hepatitis.

On the other hand, sever hepatitis such as acute hepatitis severe form and fulminant hepatitis may be caused from cases of genotype 3 positive which is considered not to be severe. In the above-described analysis, most (23 cases, 74%) of 31 severe cases are genotype 4; however, seven cases (26%) are genotype 3 (the other case is genotype 1). Therefore, attention to genotype 4 alone does not necessarily prevent severe hepatitis. Therefore, an important object of the invention is to predict the severity of the HEV genotype other than genotype 4.

### BRIEF SUMMARY OF THE INVENTION

The object of the present invention is to provide method, probe sets, and primer sets for predicting potential severity of hepatitis E.

The present invention provides, as a first embodiment, a method of predicting potential severity of hepatitis, comprising determining that hepatitis in a subject is potentially severe when it is detected that any amino acid is mutated to an amino acid of genotype-4, the any amino acid being amino acid of an amino acid sequence in a region encoded by ORF1 of an HEV genome RNA of genotype 3, and the HEV genome RNA being contained in a specimen nucleic acid taken from the subject infected with genotype-3 HEV.

### BRIEF DESCRIPTION OF THE SEVERAL VIEWS OF THE DRAWING

FIG. 1 is a schematic diagram for describing three ORFs of a hepatitis E viral genome and a protein coded by ORF1;

FIG. 2 is a schematic diagram for describing an amplification method by a LAMP method;

FIG. 3 is a schematic diagram for describing amplified products obtained by using the LAMP method;

FIG. 4 is a schematic diagram for describing the amplification method in production of nucleic acids for measurement (i.e., target nucleic acids);

FIG. 5 illustrates a phylogenetic tree focused on the evolutionary rate, which is produced using a) complete genome sequences; b) base sequences of ORF2 of the HEV genotype-3 strain;

FIG. 6A illustrates an alignment of amino acid sequences near the 1213th of the HEV genotype-3 strain and ORF1 of genotype-4 strain, namely the 239th amino acid region in a region coding for helicase; and

FIG. 6B is a diagram illustrating the alignment which is continuous from FIG. 6A.

### DETAILED DESCRIPTION OF THE INVENTION

The present invention is based on the fact that when a subject is infected with HEV in which a part of the region encoded by an open reading frame 1 (i.e., “ORF1”) of HEV 1 is mutated, hepatitis in the infected subject is potentially severe.

In this description the following terminological definitions apply: “subject” may include mammalians such as humans, pigs, wild boar, deer, mongooses, cats, rats and cows, and shellfish such as Corbicula; “specimen nucleic acid” may mean any of the nucleic acids derived from specimens obtained from the subjects, examples of such specimens including blood, serum, stool, tissue, and biopsy; and “amplification” may include nucleic acid amplification methods such as PCR and LAMP, any of the amplification methods known in themselves being usable and the LAMP method being preferable.

### [1] HEV

I. HEV Spot Genome and ORF1

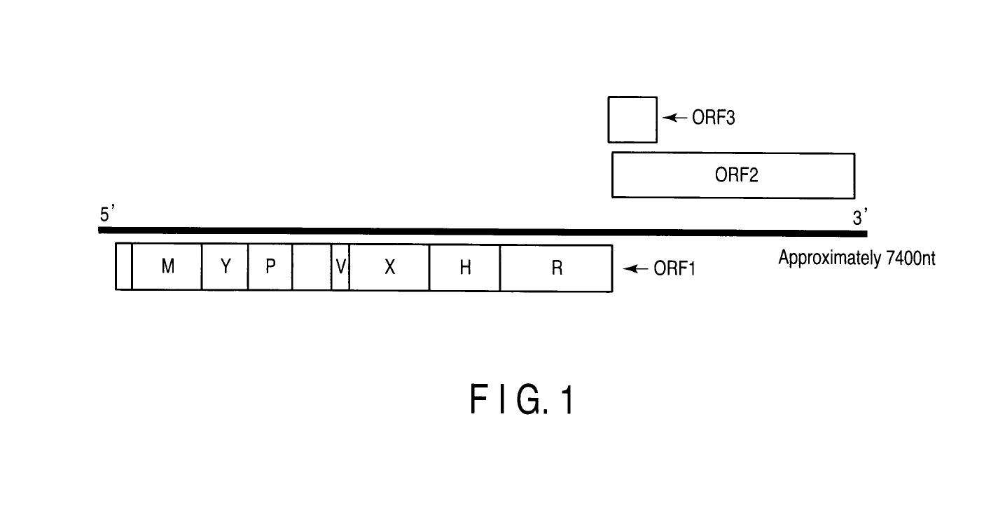

HEV is a virus which has a single strand RNA classified as the genus Hepevirus, family Hepeviridae as a genome. The full length of the genome is about 7400 bases and three ORFs are present therein (FIG. 1, cited from H. Okamoto, Genetic Variability and Evolution of Hepatitis E Virus, Virus Research 2007; 127:216-228). Among them, ORF1 is in the longest region.

ORF1 is encoded from the 27th to 5138th nucleotides and consists of a full-length 1703 amino acid. ORF1 encodes for some proteins and coding regions are present in the order of 5′ end, methyltransferase (M in FIG. 1), Y domain (Y in FIG. 1), papain-like protease (P in FIG. 1), proline-rich hinge domain (V in FIG. 1), X domain (X in FIG. 1), RNA helicase (H in FIG. 1), and RNA polymerase (R in FIG. 1).

II. Genotype of HEV and its Pathological Condition

The genotypes of HEV include genotypes 1, 2, 3, and 4 and the distribution clearly shows regional specificity. Genotype 1 is distributed in regions where hepatitis E occurs epidemically, for example, Asia and Africa. Although it is occasionally found in Japan, it is usually observed in subjects with a history of travel to Asia and Africa. Genotype 2 is a strain which has been discovered at the time of the group infection in Mexico and has been sporadically discovered in Nigeria. Genotype 3 is a strain separated from subjects with sporadic acute hepatitis which occurs in Western countries, Australia, and South Korea. The reason why many instances of genotype 3 are found in the indigenous Japanese strain is considered to be because genotype 3 was introduced through pigs which had been imported from Britain as part of a policy of increasing wealth and military power about 100 years ago (Hiroaki Okamoto, Present Situation of Hepatitis E in Japan, Liver, 2006; 47(3):379-383). Genotype 4 is a strain which is mainly observed in China and the strain is further separated from subjects with sporadic hepatitis E in Taiwan or Vietnam.

It is found that cases of genotype-4 infection tend to be severe as compared to cases of genotype-3 infection. This is apparent from the fact that the frequency of genotype 4 is increased as hepatitis is progressed from the inapparent infection group to the fulminant hepatitis group. It is also found out from the fact that significant high levels of bilirubin and transaminase at the initial visit or at peak hour are observed in cases of genotype-4 infection (Toshinori Abe, Tatsuya Aikawa, Masahiro Akabane, et al., Statistical, Epidemiological, and Virological Characteristics of Hepatitis E Virus Infection in Japan: Analysis based on 254 cases (national statistics), Liver, 2006; 47(8):384-391).

III. Severe Cases of Genotype 3

There are severe cases of genotype-3 infection. From the analysis of 254 cases of HIV infection by the MHLW Study Group, it is reported that seven (5%) out of 135 subjects with genotype 3 are severe. When hepatitis E becomes severe, serious symptoms such as severe form of acute hepatitis and fulminant hepatitis are caused. Therefore, care should be exercised in follow-up study. It is desirable that there is a method which can detect only a severe genotype-3 strain in order to previously predict pathological conditions.

IV. Characteristics of Severe Cases of Genotype 3

As shown in the following examples, characteristics of severe cases of genotype 3 have been found. It is found that when the full length of the base sequence of the HEV strain separated from a severe case of subjects with genotype-3 infection is compared to that of the HEV strain separated from a nonsevere case of the subjects, 18 amino acids are different. Among the 18 amino acids, three amino acid mutations are mutated to amino acids preserved in genotype 4.

The three amino acids are the 605th amino acid, the 978th amino acid, and the 1213th amino acid in a region encoded by ORF1 of the HEV genome RNA. As for the amino acids, the 605th amino acid of ORF1 is serine in the case of genotype 3, while it is mutated to proline of genotype 4 in the severe case. The 978th amino acid of ORF1 is isoleucine in the case of genotype 3, while it is mutated to valine of genotype 4 in the severe case. The 1213th amino acid of ORF1 is valine in the case of genotype 3, while it is mutated to alanine of genotype 4 in the severe case. Among them, the 1213th amino acid is a mutation of a site near the C terminus of the region coding for helicase.

TABLE 1

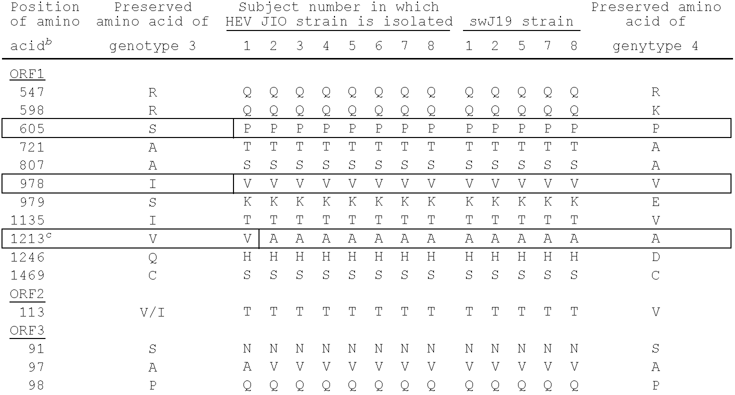

It is possible to predict that hepatitis in the subject infected with HEV in which the 605th, 978th, and 1213th amino acids are respectively the same as the amino acids of genotype 4 is potentially severe. Further, it is possible to predict that hepatitis in the subject infected with HEV in which the 605th, 978th, and 1213th amino acids are simultaneously the same as the amino acids of genotype 4 is potentially severe.

As described above, the 1213th amino acid is the mutation of the site near the C terminus of the region coding for helicase. As for Subjects 1 to 8 shown in Table 1, the 605th and 978th amino acids of HEV obtained from Subject 1 were mutated to the amino acid of genotype 4, while the 1213th amino acid was still the amino acid of genotype 3. Hepatitis did not become severe in Subject 1. Therefore, it is considered that the potential severity of hepatitis can be determined by identifying only the type of the 1213th amino acid.

On the other hand, in Subjects 1 to 8, the sites mutated to the amino acid different from the amino acid preserved in genotype 3 (referred to as a “preserved amino acid of genotype 3” in the table) were the 547th, 598th, 721st, 807th, 979th, 1135th, 1246th, and 1469th of ORF1; the 113th of ORF2; and the 91st, 97th, and 98th of ORF3.

Therefore, it can be predicted that when 547th amino acid of ORF1 of the HEV genome RNA is glutamine, the 598th amino acid is glutamine, the 605th amino acid is proline, the 721st amino acid is threonine, the 807th amino acid is serine, the 978th amino acid is valine, the 979th amino acid is lysine, the 1135th amino acid is threonine, the 1213th amino acid is alanine, the 1246th amino acid is histidine, the 1469th amino acid is serine, the 113th amino acid of ORF2 is threonine, the 91st amino acid of ORF3 is asparagine, the 97th amino acid is valine, and the 98th amino acid is glutamine, hepatitis in the subject is potentially severe.

The amino acid of HEV of genotype 3 is herein compared to that of HEV of genotype 4. In genotypes 1 and 2 in addition to genotype 3, the mutation to the amino acid of HEV of genotype 4 allows for predicting the potential severity of hepatitis in the subject infected with such a HEV.

The mutation in HEV genome is always changing. Therefore, in addition to the mutation site, it is considered that the mutation of the amino acid of any genotype except genotype 4 to the amino acid of genotype 4 is largely involved in the severity.

V. Helicase

Helicase is an enzyme with a role in unwinding the nucleic acid which is entwined while it moves along a phosphoric ester skeleton of nucleic acids. Unwinding of nucleic acid is an essential step at the time of RNA genome replication. The possibility of normal unwinding of genome RNA is considered as a key factor in accurate genome replication and genome replication at the required speed. The mutation enters a region coding for the enzyme and the amino acid of genotype 3 is replaced with the amino acid of genotype 4. This affects the ease of replication of HEV and then HEV genotype 3 exhibits behaviour like that of genotype 4. As the result, it is considered that the severity is caused.

Therefore, it is considered that the mutation of the amino acid in the region coding for helicase of genotype 3 to the amino acid of genotype 4 may be largely involved in the severity of hepatitis in a subject infected with HEV.

### [2] Method

A method of predicting the potential severity of hepatitis in a subject infected with HEV includes the steps of:

1) amplifying a specimen nucleic acid using a primer for amplification;
2) making the amplified product react with the probe for detection; and
3) predicting the potential severity of hepatitis in the subject based on the result of the reaction.

The primer for amplification may have at least a base sequence for amplifying a region including the site where at least one of the mutations is present. The probe for detection may have at least a base sequence for obtaining information for determining the presence of the mutation in the site where the mutation may be present.

The method allows for predicting the potential severity of hepatitis in the subject infected with HEV. Further, the prediction is carried out and at the same time, the type of HEV in the subject may be determined.

In that case, a nucleic acid primer for amplifying a region which contains base sequences showing characteristics of each type of HEVs and a nucleic acid probe for detecting the amplified product may be used.

I. LAMP Method

The method of amplifying a nucleic acid is preferably the LAMP method. The method is an isothermal gene amplification method and differs from the PCR method in that four or six types of primer are used. It is reported that the LAMP method is excellent in amplification efficiency, can amplify a sample in a short time, and is hardly affected by impurities in the sample as compared with the PCR method. Therefore, a trace amount of HEV in the sample can be detected in a short time by a simple pretreatment of the sample.

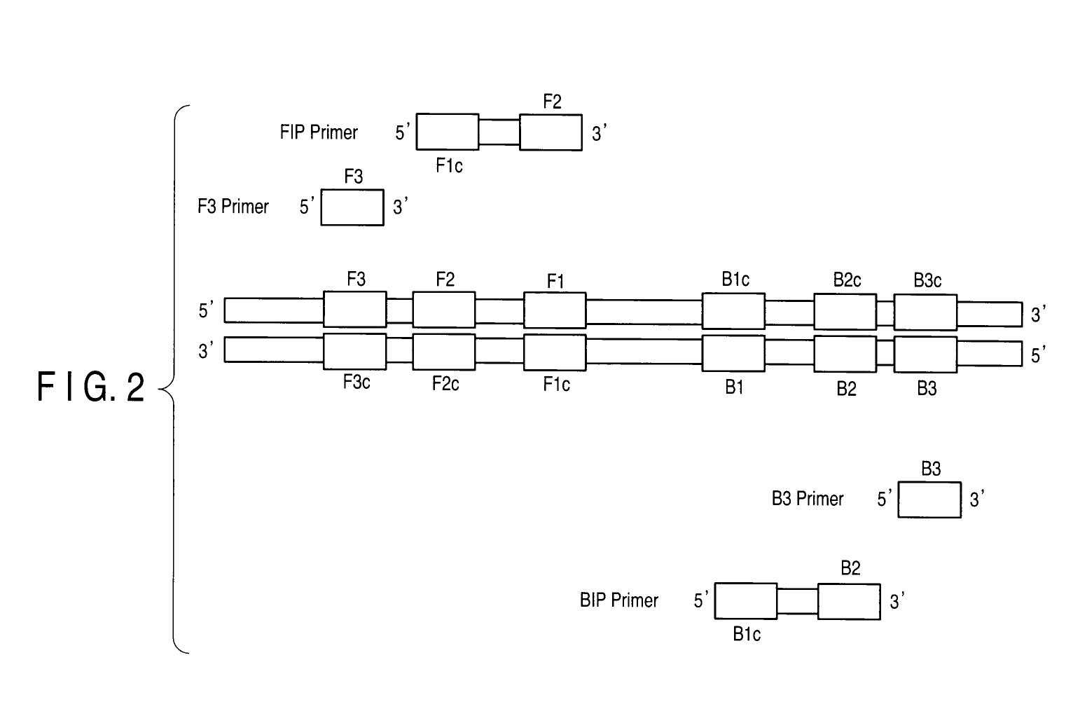

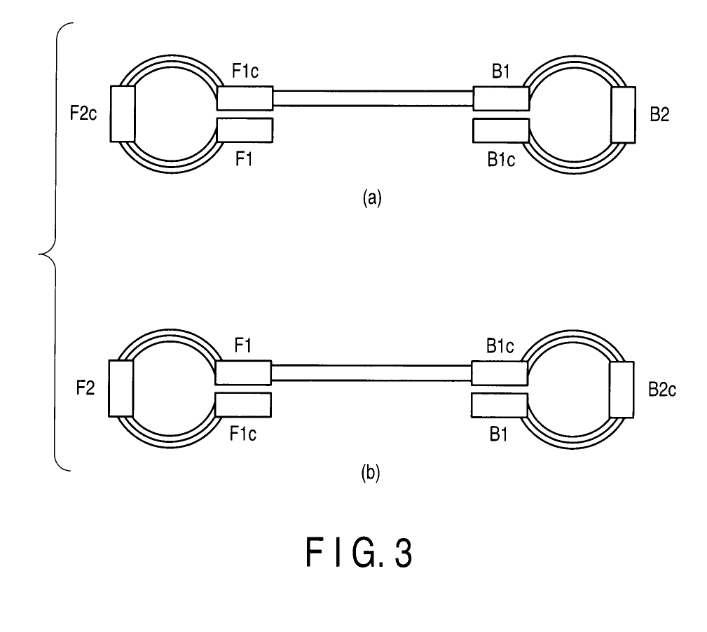

The primer design in the LAMP method as well as amplified products to be obtained will be described with reference to FIGS. 2 and 3. FIG. 2 shows the correspondence between a double-stranded DNA being detected and primers being used. Examples of the primer include an FIP primer, an F3 primer, a BIP primer, and a B3 primer. The FIP primer and the BIP primer respectively contain two regions. That is, the FIP primer contains an F1c region and an F2 region and the BIP primer contains a B1c region and a B2 region. Here, F3, F2, F1, B1c, B2c, and B3c regions are regions determined on one of the double-stranded DNA. The regions are also determined on the DNA in this order in a direction from 5′ toward 3′. Here, B3 is complementary to B3c, B2 is complementary to B2c, B1 is complementary to B1c, F1c is complementary to F1, F2c is complementary to F2, and F3c is complementary to F3.

A total of the six regions which are used as primers are hereinafter referred to as respective primer regions. When the LAMP amplification is performed using four types of primer composed of these regions, namely, the FIP, F3, BIP, and B3 primers, amplified products having a dumbbell-shaped stem-and-loop structure shown in FIG. 3 are obtained from each chain of the double-stranded DNA in FIG. 2. The amplification mechanism will not be described here. If necessary, refer to, for example, Jpn. Pat. Appln. KOKAI Publication No. 2002-186781.

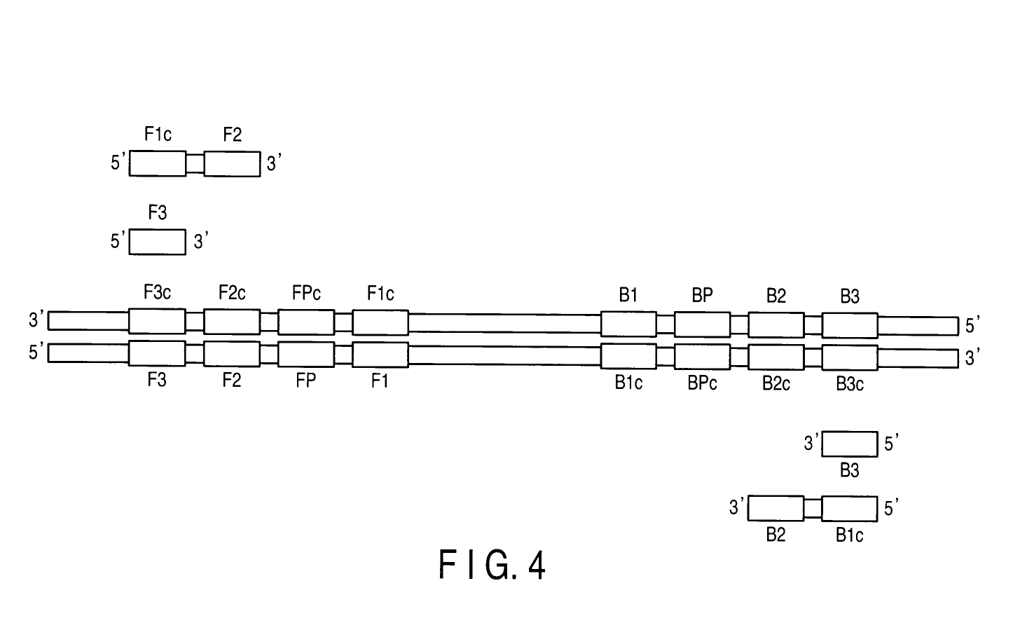

For the purpose of accelerating the amplification, a primer region may be set up in the position corresponding to any of a position between the F1 and F2 primer regions (the F2 region may be included), a position between the F2c and F1c primer regions (including the F2c region), a position between the B1 and B2 primer regions (including the B2 region) and/or a position between the B2c and B1c primer regions (including the B2c region) as shown in FIG. 4. Such a primer is referred to as a loop primer. The region is located in a single strand loop of the dumbbell structure of amplified products. When a DNA fragment complementary to the strand coexists, the amplification is smoothly progressed and the required template conformation is maintained. For that reason, it is considered that the amplification is accelerated.

As in the case of hepatitis E virus, when the genome is RNA, it is essential to have a step of synthesizing cDNA with reverse transcriptase in the conventional amplification methods. Since the LAMP method has a strand substitution activity, the amplification can be directly initiated from the extracted product by just adding reverse transcriptase to a LAMP reaction solution without a separate step for reverse transcription reaction.

The amplification reaction may be carried out by using one primer set per tube or a plurality of primer sets for various genotypes per tube. When it is necessary to identify each subtype of a plurality of hepatitis E viruses at once, it is efficient to use the latter method.

II. Extraction of Specimen and Specimen Nucleic Acid

In the method of extracting a nucleic acid component from a specimen, the liquid-liquid extraction method such as the phenol-chloroform method or the solid-liquid extraction method using a matrix may be used, but it is not limited thereto. Further, commercially available nucleic acid extraction kits; MinElute (manufactured by Qiagen) and Sumitest EX-R&D (manufactured by Medical & Biological Laboratories Co., Ltd.), and the like may be used. A nucleic acid component which is extracted by any of methods which are known in themselves may be used as the specimen nucleic acid.

III. LAMP Reaction Conditions

The extracted nucleic acid component is subjected to LAMP amplification by the method. The temperature of LAMP amplification is preferably in the range of 60 to 65° C., recommended by Eiken Chemical Co., Ltd., which developed the LAMP method.

The composition of reaction solution may have, for example, the following composition; however, it is not limited thereto.

LAMP reaction solution
Reaction solution composition of the RT-LAMP method:

20 mM Tris-HCl (pH 8.8)

10 mM KCl

8 mM MgSO₄

10 mM (NH₄)₂SO₄

0.1% Tween20

0.8 M Betaine

1.4 mM each dNTPs

When the reaction is performed using the reaction solution having the composition so as to be a total amount of 25 μl, respective primers are charged at the following concentrations.
Additive amount of primer:

FIP 40 pmol

BIP 40 pmol

F3 5 pmol

B3 5 pmol

IV. Detection Method

(1) Solid phase surface of nucleic acid probe

The detection of amplified products may be performed by detecting the presence or absence of hybridization with a nucleic acid probe. The nucleic acid probe for detecting the amplified products may or may not be solid-phased on a matrix. When the probe is solid-phased, a device in which the nucleic acid probe is solid-phased on the matrix may be used. This is generally referred to as a nucleic acid solid phase substrate, nucleic acid chip or DNA chip.

The matrix for immobilizing the nucleic acid probe may be a resin bead, magnetic bead, metal fine particle, microtiter plate, glass substrate, silicon substrate, resin substrate, and electrode substrate, but it is not limited thereto.

When the probe is not solid-phased, hybridization methods in the liquid phase which are known in themselves may be used.

(2) Matrix for solid-phase of nucleic acid probe

Inorganic insulating materials such as glass, silica glass, alumina, sapphire, forsterite, silicon carbide, silicon oxide, and silicon nitride may be used as matrix materials; however, it is not limited thereto. Further, examples of the matrix material may include organic materials such as polyethylene, ethylene, polypropylene, polyisobutylene, polymethylmethacrylate, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene acrylonitrile copolymer, acrylonitrile butadiene styrene copolymer, silicone resin, polyphenylene oxide, and polysulfone.

(3) Nucleic Acid Solid Phase Matrix

A nucleic acid solid phase matrix may be produced by methods known in themselves depending on the detection method.

(4) Hybridization

The reaction of the amplified products with the nucleic acid probe may be hybridization. The reaction may be carried out under the condition where two nucleic acids which are specifically bound are appropriately hybridized.

For example, when the hybridization reaction is performed with the amplified nucleic acid component and a gene detection electrode, it is carried out in the following manner. The reaction solution is reacted in a buffer solution with an ionic strength of 0.01 to 5 and a pH of 5 to 10. Dextran sulfate which is a hybridization enhancer, salmon sperm DNA, bovine thymus DNA, EDTA, and surfactants may be added to the solution. The extracted nucleic acid component is added thereto, which is heat-denatured at 90° C. or more. The gene detection electrode may be inserted immediately after denaturation or after rapidly cooling to 0° C. Alternatively, the hybridization reaction can be performed by dropping a solution onto a substrate. During the reaction, the reaction rate can also be increased by stirring or shaking. The reaction temperature ranges from 10 to 90° C. and the reaction time ranges from one minute to overnight. After the hybridization reaction, the electrode is taken out and washed. When washing, a buffer solution with an ionic strength of 0.01 to 5 and a pH of 5 to 10 is used.

(5) Labeling of Amplified Samples

The amplified nucleic acid samples are labeled with fluorescent dyes such as FITC, Cy3, Cy5, and rhodamine; biotin, hapten, enzymes such as oxidase and phosphatase, or electrochemically active substances such as ferrocene and quinone, or by using a second probe previously labeled with the substance described above. The presence or absence of hybridization may be detected by methods known in themselves depending on the labeled substance. Examples of the detection method include a method of detecting current, a method of detecting fluorescence, a method of detecting luminescence, and a method of detecting dye.

(6) Detection Procedure when Using Current Detection System

When the detection is performed using the electrochemically active DNA binding substance, the detection is carried out in the following procedure. The substrate is washed, followed by reaction with the DNA binding substance which selectively binds to a double strand portion formed on the surface of the electrode and electrochemical measurement. The DNA binding substance to be used herein is not particularly limited. Usable examples thereof include Hoechst 33258, acridine orange, quinacrine, donomicine, metallo-intercalator, bis-intercalator such as bisacridine, tris-intercalator, and poly-intercalator. Further, these intercalators may be modified with electrochemically active metal complexes such as ferrocene and viologen. The concentration of the DNA binding substance varies depending on the type thereof and it is generally in the range of 1 ng/ml to 1 mg/ml. In this case, a buffer solution with an ionic strength of 0.001 to 5 and a pH of 5 to 10 is used. The electrode is reacted with the DNA binding substance, followed by washing and electrochemical measurement. The electrochemical measurement is carried out using a three-electrode type (i.e., a reference electrode, a counterelectrode, and a working electrode) or a two-electrode type (i.e., a counterelectrode and a working electrode). In the measurement, a potential at which the DNA binding substance reacts electrochemically or higher is applied and a reaction current value derived from the DNA binding substance is measured. In the process, the potential may be swept at a constant speed, a potential pulse may be applied or a constant potential may be applied. In the measurement, the current and voltage are controlled using apparatuses such as potentiostats, digital multimeters, and function generators. The concentration of target genes is calculated from calibration curve based on the current values obtained. The gene detection apparatus using the gene detection electrode includes a gene extraction unit, a gene reaction unit, a DNA-binding substance reaction unit, an electrochemical measurement unit, and a washing unit.

(7) Results

When using the current detection method or other solid phase matrixes, the sequence of the amplified products can be determined based on the sequence of a probe which is solid-phased on an electrode in which a positive signal derived from a label is obtained or a fraction (i.e., region) of, for example, a well. As a result, an amino acid mutation or genotype of HEV genome present in the sample can be determined. On the basis of the determined amino acid mutation or genotype of HEV, it is determined whether HEV in the subject is potentially severe. Further, the type of HEV may be simultaneously determined.

### VI. Probes

Preferable examples of the probe to be used for identifying the amino acid of the mutation site according to the present invention are shown in Table 2.

TABLE 2

| | |
|---|---|
| SEQ ID NO: 1 | CGGYATWTCNGAT(GCN)ATYGTYAAYAAYTT |
| SEQ ID NO: 2 | CGGYATWTCNGAT(GTN)ATYGTYAAYAAYTT |
| SEQ ID NO: 3 | YGGYATYTCWCAY(ATM)ATTGTWAAYAAYTTC |
| SEQ ID NO: 4 | GGGCATCTCYGAY(CCA)ATYGTYAATAAYTT |
| SEQ ID NO: 5 | GGGTATCTCAGAT(GCC)ATTGTTAATAATTT |
| SEQ ID NO: 6 | CCGYATYTCWGAY(GCY)ATTGTTAATAACTT |

Examples thereof include:

a probe consisting of a polynucleotide which consists of a continuous base sequence of 15 to 30 by including the 14th to 16th base sequence (the portion in parentheses in Table 2) of a polynucleotide represented by SEQ ID NO:1 and/or a complementary sequence thereof; a probe consisting of a polynucleotide which consists of a continuous base sequence of 15 to 30 by including the 14th to 16th base sequence (the portion in parentheses in Table 2) of a polynucleotide represented by SEQ ID NO:2 and/or a complementary sequence thereof; and
a probe consisting of a polynucleotide which consists of a continuous base sequence of 15 to 30 by including the 14th to 16th base sequence (the portion in parentheses in Table 2) of a polynucleotide represented by SEQ ID NO:3 and/or a complementary sequence thereof.

Further, examples thereof may include: a probe consisting of a polynucleotide which consists of a continuous base sequence of 15 to 30 by including the 14th to 16th base sequence (the portion in parentheses in Table 2) of a polynucleotide represented by SEQ ID NO:4 and/or a complementary sequence thereof;

a probe consisting of a polynucleotide which consists of a continuous base sequence of 15 to 30 by including the 14th to 16th base sequence (the portion in parentheses in Table 2) of a polynucleotide represented by SEQ ID NO:5 and/or a complementary sequence thereof; and
a probe consisting of a polynucleotide which consists of a continuous base sequence of 15 to 30 by including the 14th to 16th base sequence (the portion in parentheses in Table 2) of a polynucleotide represented by SEQ ID NO:6 and/or a complementary sequence thereof.

When the probes are used, they may be used as a mixed base. Alternatively, several probes to be required may be mixed for use. When used as the mixed base, for example, the probe is used in the following manner. Y contained in the base sequence represents thymine or cytosine. The probe containing thymine and the probe containing cytosine are mixed for use. Similarly, W represents thymine or adenine and two probes containing either of them may be used together, for example, in a mixed state. M represents cytosine or adenine and two probes containing either of them may be used together, for example, in a mixed state. N represents thymine, cytosine, adenine or guanine and four probes containing any of them may be used together.

These probes are also referred to as probes for detection. The 1213th amino acid of ORF1 can be identified by hybridizing these probes with the specimen nucleic acid and detecting the presence of hybridization.

The probes related to SEQ ID NOS:1 to 3 function in identifying the 1213th amino acid of ORF1. Similarly, a probe consisting of a polynucleotide which consists of a base sequence of 15 to 30 by can be designed so that a base coding for another mutation site, namely, the 605th or 978th amino acid of ORF1 is the 14th to 16th amino acids thereof. Such a probe is also included in the scope of the present invention.

V. Primers

Examples of the primer for LAMP amplification (simply referred to as the “primer” hereinafter) which is preferably used in the present invention will be described hereinafter. The following examples of the primer include primers which amplify the site capable of identifying the genotype of HEV contained in the specimen. These primers are preferably used as a primer set.

Preferably, a first primer set consists of:

(a) an F3 primer consisting of a polynucleotide represented by SEQ ID NO:7 and/or a polynucleotide represented by SEQ ID NO:8 and/or a polynucleotide represented by a complementary sequence thereof;
a primer FIP consisting of a polynucleotide represented by SEQ ID NO:9 and/or a polynucleotide represented by SEQ ID NO:10 and/or a polynucleotide represented by a complementary sequence thereof;
a BIP primer consisting of a polynucleotide represented by SEQ ID NO:11 and/or a polynucleotide represented by SEQ ID NO:12 and/or a polynucleotide represented by a complementary sequence thereof;
a B3 primer consisting of a polynucleotide represented by SEQ ID NO:13 and/or a polynucleotide represented by SEQ ID NO:14 and/or a polynucleotide represented by a complementary sequence thereof; and
an FLc primer consisting of a polynucleotide represented by SEQ ID NO:15 and/or a polynucleotide represented by a complementary sequence thereof in order to amplify genotype 1;
(b) an F3 primer consisting of a polynucleotide represented by SEQ ID NO:16 and/or a polynucleotide represented by a complementary sequence thereof;
an FIP primer consisting of a polynucleotide represented by SEQ ID NO:17 and/or a polynucleotide represented by a complementary sequence thereof;
a BIP primer consisting of a polynucleotide represented by SEQ ID NO:18 and/or a polynucleotide represented by a complementary sequence thereof;
a B3 primer consisting of a polynucleotide represented by SEQ ID NO: 19 and/or a polynucleotide represented by a complementary sequence thereof; and
an FLc primer consisting of a polynucleotide represented by SEQ ID NO:20 and/or a polynucleotide represented by a complementary sequence thereof in order to amplify genotype 2;
(c) an F3 primer consisting of a polynucleotide represented by SEQ ID NO:21 and/or a polynucleotide represented by SEQ ID NO:22 and/or a polynucleotide represented by SEQ ID NO:23 and/or a polynucleotide represented by a complementary sequence thereof;
an FIP primer consisting of a polynucleotide represented by SEQ ID NO:24 and/or a polynucleotide represented by SEQ ID NO:25 and/or a polynucleotide represented by SEQ ID NO:26 and/or a polynucleotide represented by SEQ ID NO:27 and/or a polynucleotide represented by a complementary sequence thereof;
a BIP primer consisting of a polynucleotide represented by SEQ ID NO:28 and/or a polynucleotide represented by SEQ ID NO:29 and/or a polynucleotide represented by SEQ ID NO:30 and/or a polynucleotide represented by SEQ ID NO:31 and/or a polynucleotide represented by SEQ ID NO:32 and/or a polynucleotide represented by SEQ ID NO:33 and/or a polynucleotide represented by a complementary sequence thereof;
a B3 primer consisting of a polynucleotide represented by SEQ ID NO:34 and/or a polynucleotide represented by SEQ ID NO:35 and/or a polynucleotide represented by a complementary sequence thereof; and
an FLc primer consisting of a polynucleotide represented by SEQ ID NO:36 and/or a polynucleotide represented by SEQ ID NO:37 and/or a polynucleotide represented by a complementary sequence thereof in order to amplify genotype 3 and determine if it is a genotype-3 HEV strain which is potentially severe; and
(d) an F3 primer consisting of a polynucleotide represented by SEQ ID NO:38 and/or a polynucleotide represented by SEQ ID NO:39 and/or a polynucleotide represented by a complementary sequence thereof;
an FIP primer consisting of a polynucleotide represented by SEQ ID NO:40 and/or a polynucleotide represented by SEQ ID NO:41 and/or a polynucleotide represented by SEQ ID NO:42 and/or a polynucleotide represented by SEQ ID NO:43 and/or a polynucleotide represented by a complementary sequence thereof;
a BIP primer consisting of a polynucleotide represented by SEQ ID NO:44 and/or a polynucleotide represented by SEQ ID NO:45 and/or a polynucleotide represented by SEQ ID NO:46 and/or a polynucleotide represented by SEQ ID NO:47 and/or a polynucleotide represented by a complementary sequence thereof;
a B3 primer consisting of a polynucleotide represented by SEQ ID NO:48 and/or a polynucleotide represented by SEQ ID NO:49 and/or a polynucleotide represented by SEQ ID NO:50 and/or a polynucleotide represented by SEQ ID NO:51 and/or a polynucleotide represented by a complementary sequence thereof; and
an FLc primer consisting of a polynucleotide represented by SEQ ID NO:52 and/or a polynucleotide represented by SEQ ID NO:53 and/or a polynucleotide represented by SEQ ID NO:54 and/or a polynucleotide represented by a complementary sequence thereof in order to amplify genotype 4.

TABLE 3

| | Genotype | | | |
|---|---|---|---|---|
| Primer | to be | Name | SEQ | |
| set No. | amplified | of primer | ID NO. | Sequence |
| I | 1 | F3 | 7 | ACCACTATTATTGCCACRGC |
| | | | 8 | ACTACCATYATTGCCACGGC |
| | | FIP | 9 | CACTTCTCAGTGTGGCGCGTCGGGGCCTTATCAGTCGTC |
| | | | 10 | CACTTCTCAGTGTGGCGCGTCGWGGCCTCATCAGTCGTC |
| | | BIP | 11 | CAGGCCTGCTTCGCGAGGTATTTCGCCACCAGCAGGAAAA |
| | | | 12 | CAGGCCTGCTTCGTGAGGTACCTCGCCACCAGCAGGAAA |
| | | B3 | 13 | CGGGGAATAACWGATGGGC |
| | | | 14 | CGAGGGATAACAGATGGGC |
| | | FLc | 15 | GCAACAATGGCATGAGCYCG |
| | 2 | F3 | 16 | TAATTGCAACTGCAGATGCC |
| | | FIP | 17 | CATTTTTCAGTATGCCTAGTGCGTGGCCTCATACAGTCCTC |
| | | BIP | 18 | TCCCGGCCTGTTGCGTGACTCGCCACCCGAAAGGAAG |
| | | B3 | 19 | CGCGGAATGACCGATGGTCT |
| | | FLc | 20 | GCAACTATAGCGTGAGCCCG |
| | 3 | F3 | 21 | ACCACAATYATAGCCACGGC |
| | | | 22 | ACCACAATTATAGCTACGGC |
| | | | 23 | ACCACAATCATAGCAACGGC |
| | | FIP | 24 | CACTTCTCAGTGTGGCGRGTGGGGCCTTATTCAGTCCATC |
| | | | 25 | CACTTCTCTGTGTGRCGGGTAGGGGCCTTATCCAGTCATC |
| | | | 26 | CACCTCTCTGTGTGGCGGGTAGGGGTCTCATCCAGTCATC |
| | | | 27 | CACTTCTCTGTGTGGCGGGTAGAGGCCTTATCCAGTAATC |
| | | BIP | 28 | CCGGCCTGCTTCGTGAGGCCTCGCCACCAGCAAGGAAA |
| | | | 29 | CGGCCTGCGCGYGAGGTCCCTCTCCACCAGCAAGGAAA |
| | | | 30 | TGGTTTGCTGCGCGAGGTCCTCCCCGCCAGCAAGGAAA |
| | | | 31 | TGGTCTGCTGCGTGAGGTCCTCCCCACCAGCGAGGAAA |
| | | | 32 | CCTGGTTTGTTACGTGAGGTGCTCCCCACCAGCGAGGAA |
| | | | 33 | CCGGTTTATTGCGTGAGGTGCCTCTCCACCAGCAAGGAAA |
| | | B3 | 34 | CGAGGTATAACAGARGGGCG |
| | | | 35 | CGGGGTATCACAGAGGGGCG |
| | | FLc | 36 | GCGACTATAGCGTGAGCYCG |
| | | | 37 | GCAACTATAGCATGAGCTCG |
| | 4 | F3 | 38 | ACTACRATTATTGCCACGGC |
| | | | 39 | ACTACTATTATTGCWACGGC |
| | | FIP | 40 | CACTTCTCCGTGTGGCGGGTGTGGGCTGATCCAGTCATC |
| | | | 41 | CACTTCTCCGTGTGACGAGTGGGGGCTGATYCAGTCCTC |
| | | | 42 | CATTTCTCCGTATGACGGGTGGGGGCTGATTCAGTCATC |
| | | | 43 | CATTTCTCTGTCTGGCGAGTGGGGGCTGATTCAGTCCTC |
| | | BIP | 44 | GGGGCTCCTCCGTGAGGTGATCTGACCACCAGAAAGGAAA |
| | | | 45 | GGGGCTTCTTCGTGAGGTGATCTCGCCACCAGAAAGGAAG |
| | | | 46 | GGGACTCCTTCGTGAGGTCATCTGGCCACCAGAAAGGAAA |
| | | | 47 | CGGGGCTTCTTCGTGAGGTTACCTGGCCGCCAGAAAGGAAA |
| | | B3 | 48 | CGCCGTATGACTGATGGACG |
| | | | 49 | GGGGTACAACTGACGGGCG |
| | | | 50 | CGCGGTATGACTGATGGACG |
| | | | 51 | CGAGGTATGACTGATGGGCG |
| | | FLc | 52 | GCCACGATTGCGTGGGCTC |
| | | | 53 | GCCACAATGGCATGAGCCC |
| | | | 54 | GCCACAATAGCGTGRGCCC |

Preferably, a second primer set consists of:

(e) an F3 primer consisting of a polynucleotide represented by SEQ ID NO:55 and/or a polynucleotide represented by a complementary sequence thereof;
an FIP primer consisting of a polynucleotide represented by SEQ ID NO:56 and/or a polynucleotide represented by SEQ ID NO:57 and/or a polynucleotide represented by a complementary sequence thereof;
a BIP primer consisting of a polynucleotide represented by SEQ ID NO:58 and/or a polynucleotide represented by SEQ ID NO:59 and/or a polynucleotide represented by a complementary sequence thereof;
a B3 primer consisting of a polynucleotide represented by SEQ ID NO:60 and/or a polynucleotide represented by a complementary sequence thereof; and
a BLc primer consisting of a polynucleotide represented by SEQ ID NO:61 and/or a polynucleotide represented by a complementary sequence thereof in order to amplify genotype 1;
(f) an F3 primer consisting of a polynucleotide represented by SEQ ID NO:62 and/or a polynucleotide represented by a complementary sequence thereof;
an FIP primer consisting of a polynucleotide represented by SEQ ID NO:63 and/or a polynucleotide represented by a complementary sequence thereof;
a BIP primer consisting of a polynucleotide represented by SEQ ID NO:64 and/or a polynucleotide represented by a complementary sequence thereof;
a B3 primer consisting of a polynucleotide represented by SEQ ID NO:65 and/or a polynucleotide represented by a complementary sequence thereof; and
a BLc primer consisting of a polynucleotide represented by SEQ ID NO:66 and/or a polynucleotide represented by a complementary sequence thereof in order to amplify genotype 2;
(g) an F3 primer consisting of a polynucleotide represented by SEQ ID NO:67 and/or a polynucleotide represented by SEQ ID NO:68 and/or a polynucleotide represented by SEQ ID NO:69 and/or a polynucleotide represented by SEQ ID NO:70 and/or a polynucleotide represented by a complementary sequence thereof;
an FIP primer consisting of a polynucleotide represented by SEQ ID NO:71 and/or a polynucleotide represented by SEQ ID NO:72 and/or a polynucleotide represented by SEQ ID NO:73 and/or a polynucleotide represented by SEQ ID NO:74 and/or a polynucleotide represented by SEQ ID NO:75 and/or a polynucleotide represented by SEQ ID NO:76 and/or a polynucleotide represented by a complementary sequence thereof;
a BIP primer consisting of a polynucleotide represented by SEQ ID NO:77 and/or a polynucleotide represented by SEQ ID NO:78 and/or a polynucleotide represented by SEQ ID NO:79 and/or a polynucleotide represented by SEQ ID NO:80 and/or a polynucleotide represented by a complementary sequence thereof;
a B3 primer consisting of a polynucleotide represented by SEQ ID NO:81 and/or a polynucleotide represented by SEQ ID NO:82 and/or a polynucleotide represented by a complementary sequence thereof; and
a BLc primer consisting of a polynucleotide represented by SEQ ID NO:83 and/or a polynucleotide represented by SEQ ID NO:84 and/or a polynucleotide represented by a complementary sequence thereof in order to amplify genotype 3 and determine if it is the genotype-3 HEV strain which is potentially severe; and
(h) an F3 primer consisting of a polynucleotide represented by SEQ ID NO:85 and/or a polynucleotide represented by SEQ ID NO:86 and/or a polynucleotide represented by SEQ ID NO:87 and/or a polynucleotide represented by SEQ ID NO:88 and/or a polynucleotide represented by a complementary sequence thereof;
an FIP primer consisting of a polynucleotide represented by SEQ ID NO:89 and/or a polynucleotide represented by SEQ ID NO:90 and/or a polynucleotide represented by SEQ ID NO:91 and/or a polynucleotide represented by SEQ ID NO:92 and/or a polynucleotide represented by a complementary sequence thereof;
a BIP primer consisting of a polynucleotide represented by SEQ ID NO:93 and/or a polynucleotide represented by SEQ ID NO:94 and/or a polynucleotide represented by SEQ ID NO:95 and/or a polynucleotide represented by SEQ ID NO:96 and/or a polynucleotide represented by a complementary sequence thereof;
a B3 primer consisting of a polynucleotide represented by SEQ ID NO:97 and/or a polynucleotide represented by SEQ ID NO:98 and/or a polynucleotide represented by SEQ ID NO:99 and/or a polynucleotide represented by SEQ ID NO:100 and/or a polynucleotide represented by a complementary sequence thereof; and
a BLc primer consisting of a polynucleotide represented by SEQ ID NO:101 and/or a polynucleotide represented by SEQ ID NO:102 and/or a polynucleotide represented by SEQ ID NO:103 and/or a polynucleotide represented by a complementary sequence thereof in order to amplify genotype 4.

TABLE 4

| | Genotype | | | |
|---|---|---|---|---|
| Primer | to be | Name | SEQ | |
| set No. | amplified | of primer | ID NO. | Sequence |
| II | 1 | F3 | 55 | ACGCGCCACACTGAGAAGTG |
| | | FIP | 56 | ATTTCGCCACCAGCAGGAAAAACCTCGCGAAGCAGGCCTG |
| | | | 57 | ACCTCGCCACCAGCAGGAAGAACCTCGCGAAGCAGGCCTG |
| | | BIP | 58 | GCCCATCWGTTATTCCCCGTGGCAAGACGGCGGGAAGG |
| | | | 59 | GCCCATCTGTTATCCCTCGTGGCAAGACGGCGGGAAGG |
| | | B3 | 60 | AGCCAACTGATGGAAGGCRC |
| | | BLc | 61 | CCTGACGCCAATGTTGACAC |
| | 2 | F3 | 62 | ACTAGGCATACTGAAAAATG |
| | | FIP | 63 | CTCGCCACCCGAAAGGAAGCGGCCTGTTGCGTGAGGTG |
| | | BIP | 64 | GACCCATCGGTCATTCCGCCATGATGAAGGTGGAAACGC |
| | | B3 | 65 | AGCAAGCTGATGGAAGGCG |
| | | BLc | 66 | CCTGACCGCAATGTTGACGT |
| | 3 | F3 | 67 | ACYCGCCACACTGAGAAGTG |
| | | | 68 | ACCCGYCACACAGAGAAGTG |
| | | | 69 | ACCCGCCACACAGAGAGGTG |
| | | | 70 | ACCCGCCACACAGAGAAGTG |
| | | FIP | 71 | CCTCGCCACCAGCAAGGAAACCGGCCTGCTTCGTGAGG |
| | | | 72 | CCTCTCCACCAGCAAGGAAAGGCCTGCTGCGYGAGGTC |
| | | | 73 | CCTCCCCGCCAGCAAGGAAATGGTTTGCTGCGCGAGGT |
| | | | 74 | CCTCCCCACCAGCGAGGAAATGGTCTGCTGCGTGAGGT |
| | | | 75 | GCTCCCCACCAGCGAGGAACCTGGTTTGTTACGTGAGGT |
| | | | 76 | CCTCTCCACCAGCAAGGAAACCGGTTTATTGCGTGAGGT |
| | | BIP | 77 | CGCCCTTCTGTGATACCTCGACAGGACGGCGGGAAGGC |
| | | | 78 | GCCCCTCCGTGATACCTCGGCAAGACGGCGGGAATGC |
| | | | 79 | GCCCCTCTGTGATACCCCGGRCAAGATGGCGGGAAGGC |
| | | | 80 | GCCCCTCTGTGATACCCCGACAAGACGGCGGGAAGGC |
| | | B3 | 81 | AGCCARCTGATGGTAAGCAC |
| | | | 82 | AGCCAACTGRTGGTAAGCAC |
| | | BLc | 83 | AACCCTGATCAGAACCTCGG |
| | | | 84 | AACCCTGACCAGAACCTTGAG |
| | 4 | F3 | 85 | ACCCGCCACACGGAGAAGTG |
| | | | 86 | ACTCGTCACACGGAGAAGTG |
| | | | 87 | ACCCGTCATACGGAGAAATG |
| | | | 88 | ACTCGCCAGACAGAGAAATG |
| | | FIP | 89 | TCTGACCACCAGAAAGGAAAAGGGGCTCCTCCGTGAGGTG |
| | | | 90 | TCTCGCCACCAGAAAGGAAGAGGGGCTTCTTCGTGAGGTG |
| | | | 91 | TCTGGCCACCAGAAAGGAAAAGGGACTCCTTCGTGAGGTC |
| | | | 92 | CCTGGCCGCCAGAAAGGAAAACGGGGCTTCTTCGTGAGGTT |
| | | BIP | 93 | CGTCCATCAGTCATACCGCGGGCAAGAGGGCGGGAAGG |
| | | | 94 | CGTCCGTCAGTTATACCCCGGGCAGGAAGGGGGGAATG |
| | | | 95 | CGTCCATCAGTCATACCGCGGGCAAGAGGGTGGGAATG |
| | | | 96 | CGTCCATCAGTCATACCGCGTTGACAAGAAGGTGGAAATGC |
| | | B3 | 97 | GCCAGCTGGTGGTTAGCAC |
| | | | 98 | GCCAGCTGATGGTACGCGC |
| | | | 99 | GCAAGCTGGTGGTAAGCGC |
| | | | 100 | CGAAGCTGGTGATAGGCGC |
| | | BLc | 101 | ACAATGTTGACACACTTGAT |
| | | | 102 | ACAATGTTGATACGCTTGAT |
| | | | 103 | GCAATGTTGCCACGCTTGAT |

Examples of the preferable probe which can identify amino acids of the mutation sites contained in amplified products amplified by such primers and at the same time can identify the HEV genotype will be described hereinafter. Refer to Table 2.

Examples thereof include:

(a) a probe for discriminating genotype 1;
a probe consisting of a polynucleotide which consists of a continuous base sequence of 15 to 30 by including the 14th to 16th base sequence (the portion in parentheses in Table 2) on SEQ ID NO:4 and/or a complementary sequence thereof;
(b) a probe for discriminating genotype 2;
a probe consisting of a polynucleotide which consists of a continuous base sequence of 15 to 30 by including the 14th to 16th base sequence (the portion in parentheses in Table 2) on SEQ ID NO:5 and/or a complementary sequence thereof;
(c) a probe for discriminating genotype 3;
a probe consisting of a polynucleotide which consists of a continuous base sequence of 15 to 30 by including the 14th to 16th base sequence (the portion in parentheses in Table 2) on each of SEQ ID NOS: 1 to 3 and/or a complementary sequence thereof; and
(d) a probe for discriminating genotype 4;
a probe consisting of a polynucleotide which consists of a continuous base sequence of 15 to 30 by including the 14th to 16th base sequence (the portion in parentheses in Table 2) on SEQ ID NO:6 and/or a complementary sequence thereof.

When these probes are used, they may be used as a mixed base. Alternatively, several probes to be required may be mixed for use.

When used as the mixed base, Y contained in the base sequences of the probes represents thymine or cytosine, W represents thymine or adenine, M represents cytosine or adenine and N represents thymine, cytosine, adenine or guanine. For example, when the base sequence containing Y is used as the mixed base, the probe containing thymine and the probe containing cytosine are preferably used together.

### EXAMPLES

### Example 1

### Cluster Involved in HEV Severity Discovered in the Strain of HEV Genotype 3

### (1) Production of Phylogenetic Tree and Severe Strain Cluster

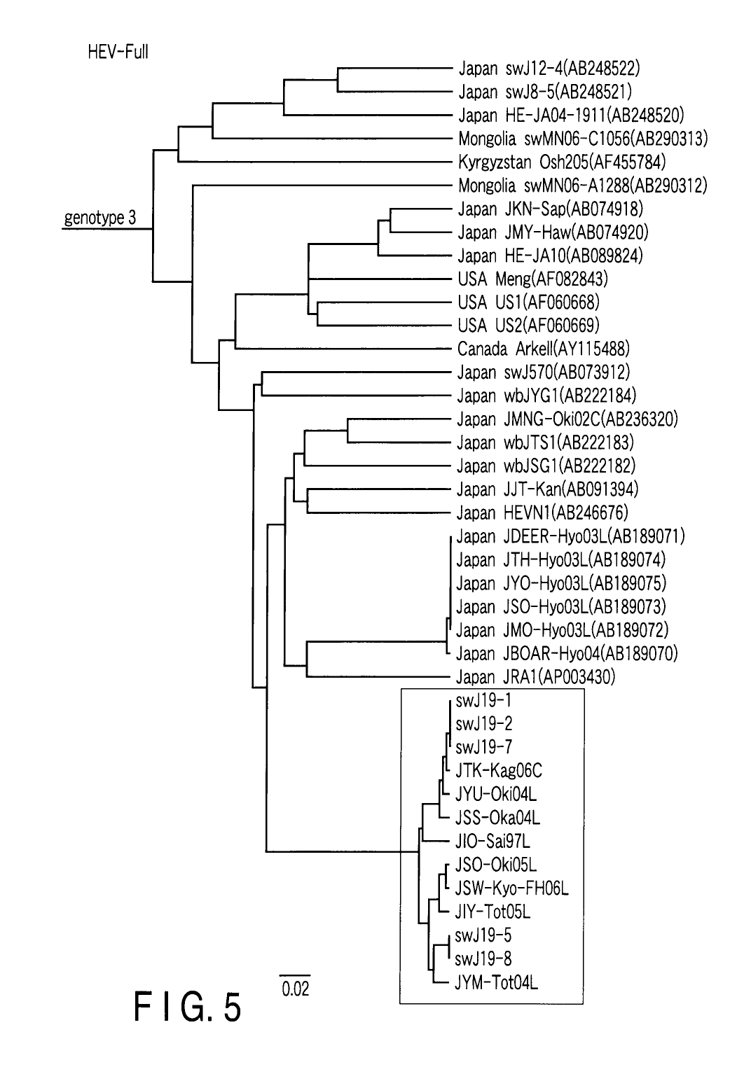

As for HEV genome sequences registered in the data bank of National Institute of Genetics (DDBJ/GenBank/EMBL databases) as well as severe cases of HEV genotype 3 and the genotype-3 strain obtained from pigs which had been gathered from all over Japan by the MHLW Study Group, the phylogenetic tree based on the evolutionary rate was produced using the full length or base sequences of ORF2 (FIG. 5). As a result, a cluster indicated by arrows in the drawing was discovered.

### (2) Structure of Severe Strain Cluster

Profiles of subjects from which the strain constituting the discovered cluster is separated

TABLE 5

| | | | | | | Degree of | |
|---|---|---|---|---|---|---|---|
| Subject | | | | Prefecture of | PT time | progression of | |
| numbe | HEV strain | Age | Gender | residence | (%)* | hepatitis | Onset time |
| 1 | JIO-Sai97L | 50 | Male | Saitama | 100 | Acute hepatitis | 1997, 4 |
| | | | | prefecture | | | |
| 2 | JYM-Tot04L | 76 | Male | Tottori | 92 | Acute hepatitis | 2004, 1 |
| | | | | prefecture | | | |
| 3 | JYU-Oki04L | 63 | Male | Okinawa | 46 | Acute hepatitis | 2004, 5 |
| | | | | prefecture | | | |
| 4 | JSS-Oka04L | 71 | Female | Okayama | 75 | Acute hepatitis | 2004, 12 |
| | | | | prefecture | | | |
| 5 | JIY-Tot05L | 65 | Male | Tottori | 34 | Acute hepatitis | 2005, 6 |
| | | | | prefecture | | severe form | |
| 6 | JSO-Oki05L | 78 | Male | Okinawa | 92 | Acute hepatitis | 2005, 7 |
| | | | | prefecture | | | |
| 7 | JTK-Kag06C | 63 | Male | Kagawa | 45 | Acute hepatitis | 2006, 3 |
| | | | | prefecture | | | |
| 8 | JSW-Kyo-FH06L | 79 | Male | Kyoto | 27 | Fulminant hepatitis | 2006, 9 |
| | | | | prefecture | | | |

*PT time: Prothrombin time. Index related to blood coagulation factor. When hepatic function is impaired, blood clotting time is lengthened. It is considered that a lower value indicates more severe hepatitis.

In a case group of genotype-3 infection, the probability of onset of severe hepatitis is seven (5%) out of 135 cases. On the other hand, in the cluster, the probability of onset is high (two [25%] out of eight cases), which is a characteristic of the cluster. Further, the prothrombin time which is a numerical value related to blood coagulation factor and is considered to show the degree of progression of hepatitis will be examined. The case in which the probability was increased from 27% to 46% was observed in Subjects 3, 5, 7, and 8. It was suggested that Subjects 5 and 8 were obviously suffering from severe hepatitis and Subjects 3 and 7 were also suffering from severe hepatitis. HEV strains separated from these cases were detected in a wide area from the Kanto region and to the west, ranging from Saitama Prefecture to Okinawa Prefecture. Since the onset time varied, it was considered that the strains were not spread mutationally, epidemically, or locally, but were spread stably in a wide area from the Kanto region and to the west. In this analysis, all of the strains (swJ19-1, 2, 5, 7, 8 in FIG. 5) obtained from pigs which were present in the same cluster of the same phylogenetic tree were detected between 2000 and 2002 in pig farms in the Kyushu region. Actually, it is estimated that three (38%) out of eight subjects ate the meat and liver of pigs or deer, which resulted in the onset of hepatitis.

Amino acid sequences of the genotype-3 and genotype-4 strains which were obtained by analysis centering around the cluster were compared over the full length (Table 1). As a result, it was found that 18 amino acids of the cluster were different from those of other strains of genotype 3. Among the 18 amino acids, 15 amino acids which are considered to be particularly important are shown in Table 1.

Among the 15 amino acids, three amino acid mutations, surrounded by a black border, were mutated to amino acids preserved in genotype 4. The three amino acids are the 605th amino acid of ORF1 which is serine in the case of genotype 3 and is mutated to proline of genotype 4, the 978th amino acid of ORF1 which is isoleucine in the case of genotype 3 and is mutated to valine of genotype 4, and the 1213th amino acid of ORF1 which is valine in the case of genotype 3 and is mutated to alanine of genotype 4. Among them, the 1213th amino acid is the mutation of the site near the C terminus of the region coding for helicase. When counted from the 1st amino acid of only helicase, the 1213th of ORF1 corresponds to the 239th of helicase.

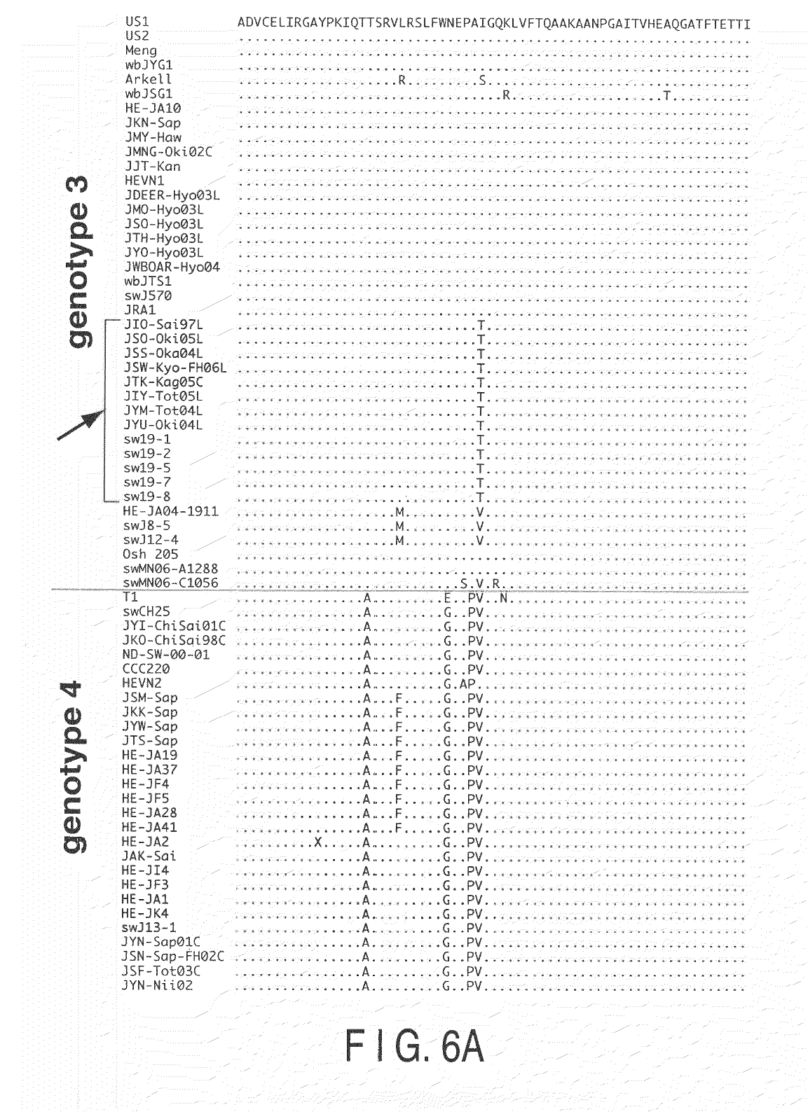

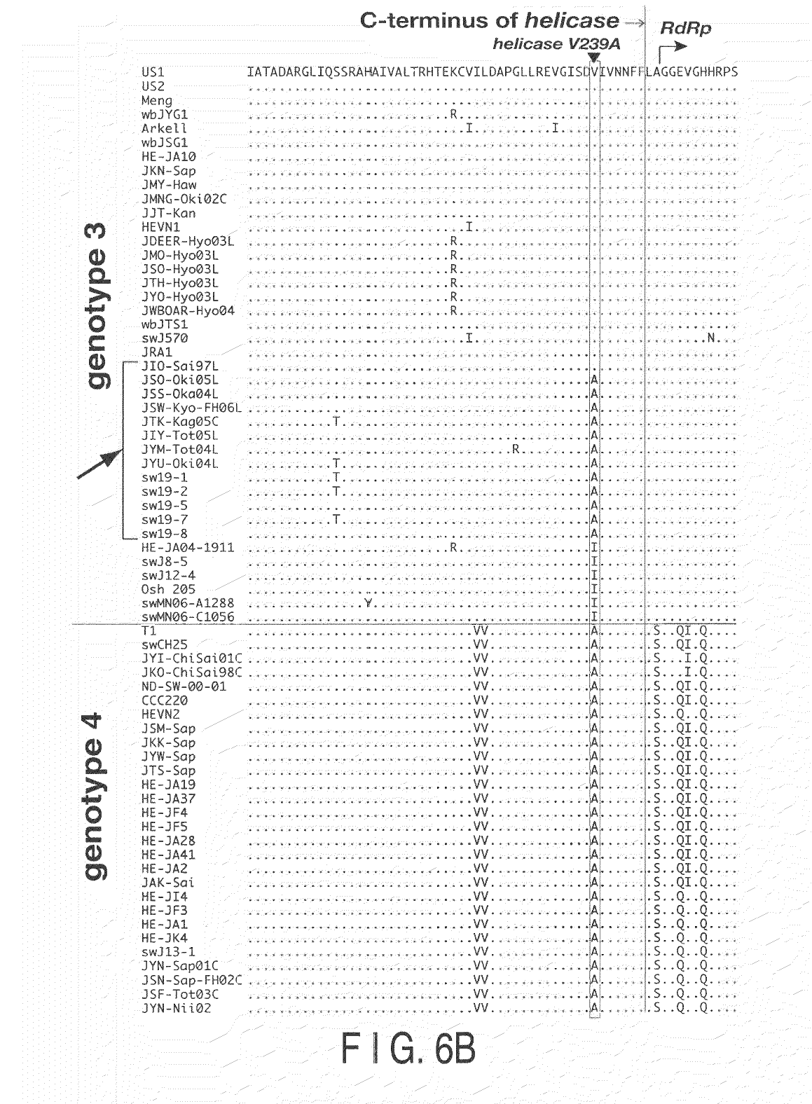

Amino acid sequences near the 1213th (the 239th of helicase) amino acid are shown along with other genotype-3 and genotype-4 strains in FIG. 6. In the case included in the cluster indicated by arrows, it was found that several sites of the amino acid sequence were different from those of the amino acid sequence of genotype 3 and they were mutated so as to be close to genotype 4.

From the above-described results, it is found that the mutation of the amino acid of genotype 3 to the amino acid of genotype 4 is important for the severity of hepatitis.

It is considered that, for example, among 15 mutations shown in Table 1, three mutations (S605P, I978V, V1213A of ORF1) which are mutated from the amino acid of genotype 3 to the amino acid of genotype 4 are particularly important. Further, it is suggested that particularly, the mutation in the 1213th amino acid of ORF1 has an important implication. Furthermore, it is suggested that the identification of all of the 15 mutations shown in Table 1 is effective for predicting the potential severity of hepatitis.

In the present example, a strain detecting the 1213th of ORF1, namely V1213A (the 239th of helicase) was established. However, even when other mutations (for example, S605P and/or 1978V of ORF1) are detected, the same effect will be obtained.

### Example 2

### Probes

The temperature of hybridization, the composition of solution, and the detection method allow for the selection of probes with various lengths. For example, when a probe set is produced using probes with a length of 30 bases, the probe set shown in the following table is considered.

TABLE 6

| | | | | | |
|---|---|---|---|---|---|
| Probe set | Mixed probe | Object genotype | V239A | SEQ ID NO. | Probe sequence |

| | | | | | |
|---|---|---|---|---|---|
| 30 | 30-1 | 3 | A | 104 | CGGCATATCGGAT(GCC)ATTGTCAACAACTT |
| | 30-2 | | V | 105 | CGGCATTTCGGAT(GTK)ATTGTCAAYAACTT |
| | | | | 106 | CGGTATTTCRGAT(GTG)ATTGTYAAYAACTT |
| | | | | 107 | CGGTATATCAGAT(GTG)ATTGTTAACAATTT |
| | 30-3 | | | 108 | CGGCATATCTGAT(GTG)ATCGTTAACAACTT |
| | | | | 109 | CGGYATATCAGAT(GTR)ATTGTCAAYAACTT |
| | | | | 110 | CGGCATATCAGAT(GTG)ATTGTCAATAACTT |
| | 30-4 | | | 111 | CGGCATATCMGAT(GTG)ATTGTCAATAACTT |
| | | | | 112 | CGGCATATCAGAT(GTG)ATTGTTAATAATTT |
| | | | | 113 | CGGCATATCGGAT(GTC)ATTGTCAACAACTT |
| | 30-5 | | I | 114 | CGGYATTTCAGAT(ATC)ATTGTAAATAATTT |
| | | | | 115 | CGGCATTTCTGAC(ATA)ATTGTTAATAACTT |
| | 30-6 | 1 | A | 116 | GGGCATCTCCGAT(GCA)ATCGTTAATAACTT |
| | | | | 117 | GGGCATCTCTGAC(GCA)ATCGTYAATAAYTT |
| | | | | 118 | CGGCATTTCTGAT(GCT)ATTGTAAATAACTT |
| | 30-7 | | | 119 | TGCGATCTCCGAT(GCA)ATCGTTAATAACTT |
| | | | | 120 | GGGCATTTCCGAT(GCA)ATCGTTAATAACTT |
| | 30-8 | 2 | A | 121 | GGGTATCTCAGAT(GCC)ATTGTTAATAATTT |
| | 30-9 | 4 | A | 122 | CGGTATCTCTGAC(GCC)ATTGTTAATAACTT |
| | 30-10 | | | 123 | CGGTATTTCTGAC(GCC)ATTGTCAATAACTT |
| | 30-11 | | | 124 | TGGCATTTCAGAT(GCC)ATTGTTAATAACTT |
| | | | | 125 | TGGTATTTCTGAT(GCT)ATTGTTAATAACTT |
| | | | | 126 | CGGCATCTCCGAT(GCC)ATAGTGAATAATTT |
| | | | | 127 | TGGTATCTCTGAC(GCC)ATAGTCAACAATTT |

In Table 6, it is not necessary to discriminate probes grouped as a mixed probe according to each type and thus they are probe groups which can be combined.

For example, when a probe set is produced using probes with a length of 27 bases, the probe set shown in Table 7 is considered.

TABLE 7

| | | | | | |
|---|---|---|---|---|---|
| Probe set | Mixed probe | Object genotype | V239A | SEQ ID NO. | Probe sequence |

| | | | | | |
|---|---|---|---|---|---|
| 27 | 27-1 | 3 | A | 128 | GGCATATCGGAT(GCC)ATTGTCAACAAC |
| | 27-2 | | V | 129 | GGCATTTCGGAT(GTK)ATTGTCAAYAAC |
| | | | | 130 | GGTATTTCRGAT(GTG)ATTGTYAAYAAC |
| | | | | 131 | GGTATATCAGAT(GTG)ATTGTTAACAAT |
| | 27-3 | | | 132 | GGCATATCTGAT(GTG)ATCGTTAACAAC |
| | | | | 133 | GGYATATCAGAT(GTR)ATTGTCAAYAAC |
| | | | | 134 | GGCATATCAGAT(GTG)ATTGTCAATAAC |
| | 27-4 | | | 135 | GGCATATCMGAT(GTG)ATTGTCAATAAC |
| | | | | 136 | GGCATATCAGAT(GTG)ATTGTTAATAAT |
| | | | | 137 | GGCATATCGGAT(GTC)ATTGTCAACAAC |
| | 27-5 | | I | 138 | GGYATTTCAGAT(ATC)ATTGTAAATAAT |
| | | | | 139 | GGCATTTCTGAC(ATA)ATTGTTAATAAC |
| | 27-6 | 1 | A | 140 | GGCATCTCCGAT(GCA)ATCGTTAATAAC |
| | | | | 141 | GGCATCTCTGAC(GCA)ATCGTYAATAAY |
| | | | | 142 | GGCATTTCTGAT(GCT)ATTGTAAATAAC |
| | 27-7 | | | 143 | GCGATCTCCGAT(GCA)ATCGTTAATAAC |
| | | | | 144 | GGCATTTCCGAT(GCA)ATCGTTAATAAC |
| | 27-8 | 2 | A | 145 | GGTATCTCAGAT(GCC)ATTGTTAATAAT |
| | 27-9 | 4 | A | 146 | GGTATCTCTGAC(GCC)ATTGTTAATAAC |
| | 27-10 | | | 147 | GGTATTTCTGAC(GCC)ATTGTCAATAAC |
| | 27-11 | | | 148 | GGCATTTCAGAT(GCC)ATTGTTAATAAC |
| | | | | 149 | GGTATTTCTGAT(GCT)ATTGTTAATAAC |
| | | | | 150 | GGCATCTCCGAT(GCC)ATAGTGAATAAT |
| | | | | 151 | GGTATCTCTGAC(GCC)ATAGTCAACAAT |

For example, when a probe set is produced using probes with a length of 23 bases, the probe set shown in the following table is considered.

TABLE 8

| | | | | | |
|---|---|---|---|---|---|
| Probe set | Mixed probe | Object genotype | V239A | SEQ ID NO. | Probe sequence |

| | | | | | |
|---|---|---|---|---|---|
| 23 | 23-1 | 3 | A | 152 | CATATCGGAT(GCC)ATTGTCAACA |
| | 23-2 | | V | 153 | CATTTCGGAT(GTK)ATTGTCAAYA |
| | | | | 154 | TATTTCRGAT(GTG)ATTGTYAAYA |
| | | | | 155 | TATATCAGAT(GTG)ATTGTTAACA |
| | 23-3 | | | 156 | CATATCTGAT(GTG)ATCGTTAACA |
| | | | | 157 | YATATCAGAT(GTR)ATTGTCAAYA |
| | | | | 158 | CATATCAGAT(GTG)ATTGTCAATA |
| | 23-4 | | | 159 | CATATCMGAT(GTG)ATTGTCAATA |
| | | | | 160 | CATATCAGAT(GTG)ATTGTTAATA |
| | | | | 161 | CATATCGGAT(GTC)ATTGTCAACA |
| | 23-5 | | I | 162 | YATTTCAGAT(ATC)ATTGTAAATA |
| | | | | 163 | CATTTCTGAC(ATA)ATTGTTAATA |
| | 23-6 | 1 | A | 164 | CATCTCCGAT(GCA)ATCGTTAATA |
| | | | | 165 | CATCTCTGAC(GCA)ATCGTYAATA |
| | | | | 166 | CATTTCTGAT(GCT)ATTGTAAATA |
| | 23-7 | | | 167 | GATCTCCGAT(GCA)ATCGTTAATA |
| | | | | 168 | CATTTCCGAT(GCA)ATCGTTAATA |
| | 23-8 | 2 | A | 169 | TATCTCAGAT(GCC)ATTGTTAATA |
| | 23-9 | 4 | A | 170 | TATCTCTGAC(GCC)ATTGTTAATA |
| | 23-10 | | | 171 | TATTTCTGAC(GCC)ATTGTCAATA |
| | 23-11 | | | 172 | CATTTCAGAT(GCC)ATTGTTAATA |
| | | | | 173 | TATTTCTGAT(GCT)ATTGTTAATA |
| | | | | 174 | CATCTCCGAT(GCC)ATAGTGAATA |
| | | | | 175 | TATCTCTGAC(GCC)ATAGTCAACA |

For example, when a probe set is produced using probes with a length of 20 bases, the probe set shown in the following table is considered.

TABLE 9

| | | | | | |
|---|---|---|---|---|---|
| Probe set | Mixed probe | Object genotype | V239A | SEQ ID NO. | Probe sequence |

| | | | | | |
|---|---|---|---|---|---|
| 20 | 20-1 | 3 | A | 176 | TATCGGAT(GCC)ATTGTCAAC |
| | 20-2 | | V | 177 | TTTCGGAT(GTK)ATTGTCAAY |
| | | | | 178 | TTTCRGAT(GTG)ATTGTYAAY |
| | | | | 179 | TATCAGAT(GTG)ATTGTTAAC |
| | 20-3 | | | 180 | TATCTGAT(GTG)ATCGTTAAC |
| | | | | 181 | TATCAGAT(GTR)ATTGTCAAY |
| | | | | 182 | TATCAGAT(GTG)ATTGTCAAT |
| | 20-4 | | | 183 | TATCMGAT(GTG)ATTGTCAAT |
| | | | | 184 | TATCAGAT(GTG)ATTGTTAAT |
| | | | | 185 | TATCGGAT(GTC)ATTGTCAAC |
| | 20-5 | | I | 186 | TTTCAGAT(ATC)ATTGTAAAT |
| | | | | 187 | TTTCTGAC(ATA)ATTGTTAAT |
| | 20-6 | 1 | A | 188 | TCTCCGAC(GCA)ATCGTCAAT |
| | | | | 189 | TCTCTGAC(GCA)ATCGTTAAT |
| | | | | 190 | TTTCTGAT(GCT)ATTGTAAAT |
| | 20-7 | | | 191 | TCTCCGAT(GCA)ATCGTTAAT |
| | | | | 192 | TTTCCGAT(GCA)ATCGTTAAT |
| | 20-8 | 2 | A | 193 | TCTCAGAT(GCC)ATTGTTAAT |
| | 20-9 | 4 | A | 194 | TCTCTGAC(GCC)ATTGTTAAT |
| | 20-10 | | | 195 | TTTCTGAC(GCC)ATTGTCAAT |
| | 20-11 | | | 196 | TTTCAGAT(GCC)ATTGTTAAT |
| | | | | 197 | TTTCTGAT(GCT)ATTGTTAAT |
| | | | | 198 | TCTCCGAT(GCC)ATAGTGAAT |
| | | | | 199 | TCTCTGAC(GCC)ATAGTCAAC |

The use of the primer for LAMP amplification and/or the probe sets allows for efficiently and accurately predicting the potential severity of hepatitis in the subject infected with HEV.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of predicting potential severity of hepatitis, comprising: determining that hepatitis in a subject is potentially severe when it is detected that any amino acid is mutated to an amino acid of genotype 4, said any amino acid being amino acid of an amino acid sequence in a region encoded by ORF1 of an HEV genome RNA of genotype 3, and said HEV genome RNA being contained in a specimen nucleic acid taken from the subject infected with genotype-3 HEV.

2. The method according to claim 1, further comprising: determining that hepatitis in a subject is potentially severe when a 1213th amino acid is alanine, said 1213th amino acid being in the region encoded by ORF1 of the HEV genome RNA, and said HEV genome RNA being contained in a specimen nucleic acid taken from the subject infected with HEV.

3. The method according to claim 1, further comprising: determining that hepatitis in a subject is potentially severe when a 605th amino acid is proline, said 605th amino acid being in the region encoded by ORF1 of the HEV genome RNA, and said HEV genome RNA being contained in the specimen nucleic acid taken from the subject infected with HEV.

4. The method according to claim 1, further comprising: determining that hepatitis in a subject is potentially severe when a 978th amino acid in the region encoded by ORF1 of the HEV genome RNA which is contained in the specimen nucleic acid taken from the subject infected with HEV is valine, said 978th amino acid being in the region encoded by ORF1 of the HEV genome RNA, and said HEV genome RNA being contained in the specimen nucleic acid taken from the subject infected with HEV.

5. The method according to claim 1, further comprising: determining that hepatitis in a subject is potentially severe when a 605th amino acid is proline, a 978th amino acid is valine, and a 1213th amino acid is alanine, said 605th, 978th and 1213th amino acid being in the region encoded by ORF1 of the HEV genome RNA, said HEV genome RNA being contained in the specimen nucleic acid taken from the subject infected with HEV.

6. The method according to claim 1, further comprising: determining that hepatitis in a subject is potentially severe when a 547th amino acid is glutamine, a 598th amino acid is glutamine, a 605th amino acid is proline, a 721st amino acid is threonine, a 807th amino acid is serine, a 978th amino acid is valine, a 979th amino acid is lysine, a 1135th amino acid is threonine, a 1213th amino acid is alanine, a 1246th amino acid is histidine, a 1469th amino acid is serine, a 113th amino acid is threonine, a 91st amino acid is asparagine, a 97th amino acid is valine, and a 98th amino acid is glutamine, said 547th, 598th, 605th, 721st, 807th, 978th, 979th, 1135th, 1213th, 1246th and 1469th amino acid being in the region encoded by ORF1 of the HEV genome RNA, said 113th amino acid being in a region encoded by ORF2 of the HEV genome RNA, said 91st, 97th and 98th amino acid being in a region encoded by ORF3 of the HEV genome RNA, said the HEV genome RNA being contained in the specimen nucleic acid taken from the subject infected with HEV.

7. The method according to claim 1, further comprising:
reacting arbitrarily amplified specimen nucleic acids with a probe set consisting of the following probes:
(a) a probe consisting of a polynucleotide which consists of a continuous base sequence of 15 to 30 by on SEQ ID NO: 1 including 14th to 16th base sequence thereupon and/or a complementary sequence thereof;
(b) a probe consisting of a polynucleotide which consists of a continuous base sequence of 15 to 30 on
SEQ ID NO: 2 by including the 14th to 16th base sequence thereupon and/or a complementary sequence thereof; and
(c) a probe consisting of a polynucleotide which consists of a continuous base sequence of 15 to 30 by on SEQ ID NO: 3 including the 14th to 16th base sequence thereupon and/or a complementary sequence thereof,
wherein Y contained in the base sequences of the probes represents thymine and cytosine, W represents thymine and adenine, M represents cytosine or adenine, and N represents thymine, cytosine, adenine, and guanine, and the probes are used as a mixed base.

8. The method according to claim 1, comprising:
reacting arbitrarily amplified specimen nucleic acids with a probe set consisting of the following probes:
(a) a probe consisting of a polynucleotide which consists of a continuous base sequence of 15 to 30 by on SEQ ID NO: 4 including 14th to 16th base sequence thereupon and/or a complementary sequence thereof;
(b) a probe consisting of a polynucleotide which consists of a continuous base sequence of 15 to 30 by on SEQ ID NO: 5 including the 14th to 16th base sequence thereupon and/or a complementary sequence thereof;
(c) a probe consisting of a polynucleotide which consists of a continuous base sequence of 15 to 30 by on SEQ ID NO: 1, 2 or 3 including the 14th to 16th base sequence thereupon and/or a complementary sequence thereof; and
(d) a probe consisting of a polynucleotide which consists of a continuous base sequence of 15 to 30 by on SEQ ID NO: 6 including the 14th to 16th base sequence thereupon and/or a complementary sequence thereof,
wherein Y contained in respective sequences represents thymine and cytosine, W represents thymine and adenine, M represents cytosine and adenine, and N represents thymine, cytosine, adenine, and guanine, and the probes are used as a mixed base.

9. The method according to claim 1, comprising:
amplifying a specimen nucleic acid using a primer for LAMP amplification;
reacting the amplified product with a probe for detection;
identifying the type of HEV based on the result of the reaction; and
determining if it is a genotype-3 HEV strain which is potentially severe,
wherein the primer for LAMP amplification is at least one selected from the group consisting of a first primer set and a second primer set and the first primer set and the second primer set consist of an F3 primer, an FIP primer, a BIP primer, a B3 primer, and an FLc primer,
in the first primer set in order to amplify HEV of genotype 1,
the F3 primer consists of a polynucleotide represented by SEQ ID NO: 7 and/or SEQ ID NO: 8 and/or a complementary sequence thereof,
the FIP primer consists of a polynucleotide represented by SEQ ID NO: 9 and/or SEQ ID NO: 10 and/or a complementary sequence thereof,
the BIP primer consists of a polynucleotide represented by SEQ ID NO: 11 and/or SEQ ID NO: 12 and/or a complementary sequence thereof,
the B3 primer consists of a polynucleotide represented by SEQ ID NO: 13 and/or SEQ ID NO: 14 and/or a complementary sequence thereof,
the FLc primer consists of a polynucleotide represented by SEQ ID NO: 15 and/or a complementary sequence thereof,
in order to amplify HEV of genotype 2,
the F3 primer consists of a polynucleotide represented by SEQ ID NO: 16 and/or a complementary sequence thereof,
the FIP primer consists of a polynucleotide represented by SEQ ID NO: 17 and/or a complementary sequence thereof,
the BIP primer consists of a polynucleotide represented by SEQ ID NO: 18 and/or a complementary sequence thereof,
the B3 primer consists of a polynucleotide represented by SEQ ID NO: 19 and/or a complementary sequence thereof,
the FLc primer consists of a polynucleotide represented by SEQ ID NO: 20 and/or a complementary sequence thereof,
in order to amplify HEV of genotype 3 and determine if it is the genotype-3 HEV strain which is potentially severe,
the F3 primer consists of a polynucleotide represented by SEQ ID NO: 21 and/or SEQ ID NO: 22 and/or SEQ ID NO: 23 and/or a complementary sequence thereof,
the FIP primer consists of a polynucleotide represented by SEQ ID NO: 24 and/or SEQ ID NO: 25 and/or SEQ ID NO: 26 and/or SEQ ID NO: 27 and/or a complementary sequence thereof,
the BIP primer consists of a polynucleotide represented by SEQ ID NO: 28 and/or SEQ ID NO: 29 and/or SEQ ID NO: 30 and/or SEQ ID NO: 31 and/or SEQ ID NO: 32 and/or SEQ ID NO: 33 and/or a complementary sequence thereof,
the B3 primer consists of a polynucleotide represented by SEQ ID NO: 34 and/or SEQ ID NO: 35 and/or a complementary sequence thereof,
the FLc primer consists of a polynucleotide represented by SEQ ID NO: 36 and/or SEQ ID NO: 37 and/or a complementary sequence thereof,
in order to amplify HEV of genotype 4,
the F3 primer consists of a polynucleotide represented by SEQ ID NO: 38 and/or SEQ ID NO: 39 and/or a complementary sequence thereof,
the FIP primer consists of a polynucleotide represented by SEQ ID NO: 40 and/or SEQ ID NO: 41 and/or SEQ ID NO: 42 and/or SEQ ID NO: 43 and/or a complementary sequence thereof,
the BIP primer consists of a polynucleotide represented by SEQ ID NO: 44 and/or SEQ ID NO: 45 and/or SEQ ID NO: 46 and/or SEQ ID NO: 47 and/or a complementary sequence thereof,
the B3 primer consists of a polynucleotide represented by SEQ ID NO: 48 and/or SEQ ID NO: 49 and/or SEQ ID NO: 50 and/or SEQ ID NO: 51 and/or a complementary sequence thereof,
the FLc primer consists of a polynucleotide represented by SEQ ID NO: 52 and/or SEQ ID NO: 53 and/or SEQ ID NO: 54 and/or a complementary sequence thereof,
in the second primer set in order to amplify HEV of genotype 1,
the F3 primer consists of a polynucleotide represented by SEQ ID NO: 55 and/or a complementary sequence thereof,
the BIP primer consists of a polynucleotide represented by SEQ ID NO: 56 and/or SEQ ID NO: 57 and/or a complementary sequence thereof,
the BIP primer consists of a polynucleotide represented by SEQ ID NO: 58 and/or SEQ ID NO: 59 and/or a complementary sequence thereof,
the B3 primer consists of a polynucleotide represented by SEQ ID NO: 60 and/or a complementary sequence thereof,
the BLc primer consists of a polynucleotide represented by SEQ ID NO: 61 and/or a complementary sequence thereof,
in order to amplify HEV of genotype 2,
the F3 primer consists of a polynucleotide represented by SEQ ID NO: 62 and/or a complementary sequence thereof,
the FIP primer consists of a polynucleotide represented by SEQ ID NO: 63 and/or a complementary sequence thereof,
the BIP primer consists of a polynucleotide represented by SEQ ID NO: 64 and/or a complementary sequence thereof,
the B3 primer consists of a polynucleotide represented by SEQ ID NO: 65 and/or a complementary sequence thereof,
the BLc primer consists of a polynucleotide represented by SEQ ID NO: 66 and/or a complementary sequence thereof,
in order to amplify HEV of genotype 3 and determine if it is the genotype-3 HEV strain which is potentially severe,
the F3 primer consists of a polynucleotide represented by SEQ ID NO: 67 and/or SEQ ID NO: 68 and/or SEQ ID NO: 69 and/or SEQ ID NO: 70 and/or a complementary sequence thereof,
the FIP primer consists of a polynucleotide represented by SEQ ID NO: 71 and/or SEQ ID NO: 72 and/or SEQ ID NO: 73 and/or SEQ ID NO: 74 and/or SEQ ID NO: 75 and/or SEQ ID NO: 76 and/or a complementary sequence thereof,
the BIP primer consists of a polynucleotide represented by SEQ ID NO: 77 and/or SEQ ID NO: 78 and/or SEQ ID NO: 79 and/or SEQ ID NO: 80 and/or a complementary sequence thereof,
the B3 primer consists of a polynucleotide represented by SEQ ID NO: 81 and/or SEQ ID NO: 82 and/or a complementary sequence thereof,
the BLc primer consists of a polynucleotide represented by SEQ ID NO: 83 and/or SEQ ID NO: 84 and/or a complementary sequence thereof,
in order to amplify HEV of genotype 4,
the F3 primer consists of a polynucleotide represented by SEQ ID NO: 85 and/or SEQ ID NO: 86 and/or SEQ ID NO: 87 and/or SEQ ID NO: 88 and/or a complementary sequence thereof,
the FIP primer consists of a polynucleotide represented by SEQ ID NO: 89 and/or SEQ ID NO: 90 and/or SEQ ID NO: 91 and/or SEQ ID NO: 92 and/or a complementary sequence thereof,
the BIP primer consists of a polynucleotide represented by SEQ ID NO: 93 and/or SEQ ID NO: 94 and/or SEQ ID NO: 95 and/or SEQ ID NO: 96 and/or a complementary sequence thereof,
the B3 primer consists of a polynucleotide represented by SEQ ID NO: 97 and/or SEQ ID NO: 98 and/or SEQ ID NO: 99 and/or SEQ ID NO: 100 and/or a complementary sequence thereof, and
the BLc primer consists of a polynucleotide represented by SEQ ID NO: 101 and/or SEQ ID NO: 102 and/or SEQ ID NO: 103 and/or a complementary sequence thereof.

10. A probe set to be used in the method according to claim 7, comprising the following probes:
(a) a probe consisting of a polynucleotide which consists of a continuous base sequence of 15 to 30 by including the 14th to 16th base sequence on SEQ ID NO: 1 and/or a complementary sequence thereof;
(b) a probe consisting of a polynucleotide which consists of a continuous base sequence of 15 to 30 by on SEQ ID NO: 2 including the 14th to 16th base sequence thereupon and/or a complementary sequence thereof; and
(c) a probe consisting of a polynucleotide which consists of a continuous base sequence of 15 to 30 by on SEQ ID NO: 3 including the 14th to 16th base sequence thereupon and/or a complementary sequence thereof,
wherein Y contained in the base sequences of the probes represents thymine and cytosine, W represents thymine and adenine, M represents cytosine and adenine, and N represents thymine, cytosine, adenine, and guanine, and the probes are used as a mixed base.

11. A probe set to be used in the method according to claim 8, comprising the following probes:
(a) a probe consisting of a polynucleotide which consists of a continuous base sequence of 15 to 30 by on SEQ ID NO: 4 including the 14th to 16th base sequence thereupon and/or a complementary sequence thereof;
(b) a probe consisting of a polynucleotide which consists of a continuous base sequence of 15 to 30 by on SEQ ID NO: 5 including the 14th to 16th base sequence thereupon and/or a complementary sequence thereof;
(c) a probe consisting of a polynucleotide which consists of a continuous base sequence of 15 to 30 by on SEQ ID NO: 1, 2 or 3 including the 14th to 16th base sequence thereupon and/or a complementary sequence thereof; and
(d) a probe consisting of a polynucleotide which consists of a continuous base sequence of 15 to 30 by on SEQ ID NO: 6 including the 14th to 16th base sequence thereupon and/or a complementary sequence thereof,
wherein Y contained in the base sequences of the probes represents thymine or cytosine, W represents thymine or adenine, M represents cytosine or adenine, and N represents thymine, cytosine, adenine or guanine, and the probes are used as a mixed base.

12. A primer set to be used in the method according to claim 9, comprising a first primer and a second primer,
wherein the first primer set consists of:
(a) an F3 primer consisting of a polynucleotide represented by SEQ ID NO: 7 and/or SEQ ID NO: 8 and/or a complementary sequence thereof;
an FIP primer consisting of a polynucleotide represented by SEQ ID NO: 9 and/or SEQ ID NO: 10 and/or a complementary sequence thereof;
a BIP primer consisting of a polynucleotide represented by SEQ ID NO: 11 and/or SEQ ID NO: 12 and/or a complementary sequence thereof;
a B3 primer consisting of a polynucleotide represented by SEQ ID NO: 13 and/or SEQ ID NO: 14 and/or a complementary sequence thereof; and
an FLc primer consisting of a polynucleotide represented by SEQ ID NO: 15 and/or a complementary sequence thereof in order to amplify HEV of genotype 1;
(b) an F3 primer consisting of a polynucleotide represented by SEQ ID NO: 16 and/or a complementary sequence thereof;
an FIP primer consisting of a polynucleotide represented by SEQ ID NO: 17 and/or a complementary sequence thereof;
a BIP primer consisting of a polynucleotide represented by SEQ ID NO: 18 and/or a complementary sequence thereof;
a B3 primer consisting of a polynucleotide represented by SEQ ID NO: 19 and/or a complementary sequence thereof; and
an FLc primer consisting of a polynucleotide represented by SEQ ID NO: 20 and/or a complementary sequence thereof in order to amplify HEV of genotype 2;
(c) an F3 primer consisting of a polynucleotide represented by SEQ ID NO: 21 and/or SEQ ID NO: 22 and/or SEQ ID NO: 23 and/or a complementary sequence thereof;
an FIP primer consisting of a polynucleotide represented by SEQ ID NO: 24 and/or SEQ ID NO: 25 and/or SEQ ID NO: 26 and/or SEQ ID NO: 27 and/or a complementary sequence thereof;
a BIP primer consisting of a polynucleotide represented by SEQ ID NO: 28 and/or SEQ ID NO: 29 and/or SEQ ID NO: 30 and/or SEQ ID NO: 31 and/or SEQ ID NO: 32 and/or SEQ ID NO: 33 and/or a complementary sequence thereof;
a B3 primer consisting of a polynucleotide represented by SEQ ID NO: 34 and/or SEQ ID NO: 35 and/or a complementary sequence thereof; and
an FLc primer consisting of a polynucleotide represented by SEQ ID NO: 36 and/or SEQ ID NO: 37 and/or a complementary sequence thereof in order to amplify HEV of genotype 3 and determine if it is the genotype-3 HEV strain which is potentially severe; and
(d) an F3 primer consisting of a polynucleotide represented by SEQ ID NO: 38 and/or SEQ ID NO: 39 and/or a complementary sequence thereof;
an FIP primer consisting of a polynucleotide represented by SEQ ID NO: 40 and/or SEQ ID NO: 41 and/or SEQ ID NO: 42 and/or SEQ ID NO: 43 and/or a complementary sequence thereof;
a BIP primer consisting of a polynucleotide represented by SEQ ID NO: 44 and/or SEQ ID NO: 45 and/or SEQ ID NO: 46 and/or SEQ ID NO: 47 and/or a complementary sequence thereof;
a B3 primer consisting of a polynucleotide represented by SEQ ID NO: 48 and/or SEQ ID NO: 49 and/or SEQ ID NO: 50 and/or SEQ ID NO: 51 and/or a complementary sequence thereof; and
an FLc primer consisting of a polynucleotide represented by SEQ ID NO: 52 and/or SEQ ID NO: 53 and/or SEQ ID NO: 54 and/or a complementary sequence thereof in order to amplify HEV of genotype 4,
wherein the second primer set consists of:
(e) an F3 primer consisting of a polynucleotide represented by SEQ ID NO: 55 and/or a complementary sequence thereof;
an FIP primer consisting of a polynucleotide represented by SEQ ID NO: 56 and/or SEQ ID NO: 57 and/or a complementary sequence thereof;
a BIP primer consisting of a polynucleotide represented by SEQ ID NO: 58 and/or SEQ ID NO: 59 and/or a complementary sequence thereof;
a B3 primer consisting of a polynucleotide represented by SEQ ID NO: 60 and/or a complementary sequence thereof; and
a BLc primer consisting of a polynucleotide represented by SEQ ID NO: 61 and/or a complementary sequence thereof in order to amplify HEV of genotype 1;
(f) an F3 primer consisting of a polynucleotide represented by SEQ ID NO: 62 and/or a complementary sequence thereof;
an FIP primer consisting of a polynucleotide represented by SEQ ID NO: 63 and/or a complementary sequence thereof;
a BIP primer consisting of a polynucleotide represented by SEQ ID NO: 64 and/or a complementary sequence thereof;
a B3 primer consisting of a polynucleotide represented by SEQ ID NO: 65 and/or a complementary sequence thereof; and
a BLc primer consisting of a polynucleotide represented by SEQ ID NO: 66 and/or a complementary sequence thereof in order to amplify HEV of genotype 2;
(g) an F3 primer consisting of a polynucleotide represented by SEQ ID NO: 67 and/or SEQ ID NO: 68 and/or SEQ ID NO: 69 and/or SEQ ID NO: 70 and/or a complementary sequence thereof;
an FIP primer consisting of a polynucleotide represented by SEQ ID NO: 71 and/or SEQ ID NO: 72 and/or SEQ ID NO: 73 and/or SEQ ID NO: 74 and/or SEQ ID NO: 75 and/or SEQ ID NO: 76 and/or a complementary sequence thereof;
a BIP primer consisting of a polynucleotide represented by SEQ ID NO: 77 and/or SEQ ID NO: 78 and/or SEQ ID NO: 79 and/or SEQ ID NO: 80 and/or a complementary sequence thereof;
a B3 primer consisting of a polynucleotide represented by SEQ ID NO: 81 and/or SEQ ID NO: 82 and/or a complementary sequence thereof; and
a BLc primer consisting of a polynucleotide represented by SEQ ID NO: 83 and/or SEQ ID NO: 84 and/or a complementary sequence thereof in order to amplify HEV of genotype 3 and determine if it is the genotype-3 HEV strain which is potentially severe; and
(h) an F3 primer consisting of a polynucleotide represented by SEQ ID NO: 85 and/or SEQ ID NO: 86 and/or SEQ ID NO: 87 and/or SEQ ID NO: 88 and/or a complementary sequence thereof;
an FIP primer consisting of a polynucleotide represented by SEQ ID NO: 89 and/or SEQ ID NO: 90 and/or SEQ ID NO: 91 and/or SEQ ID NO: 92 and/or a complementary sequence thereof;
a BIP primer consisting of a polynucleotide represented by SEQ ID NO: 93 and/or SEQ ID NO: 94 and/or SEQ ID NO: 95 and/or SEQ ID NO: 96 and/or a complementary sequence thereof;
a B3 primer consisting of a polynucleotide represented by SEQ ID NO: 97 and/or SEQ ID NO: 98 and/or SEQ ID NO: 99 and/or SEQ ID NO: 100 and/or a complementary sequence thereof; and
a BLc primer consisting of a polynucleotide represented by SEQ ID NO: 101 and/or SEQ ID NO: 102 and/or SEQ ID NO: 103 and/or a complementary sequence thereof in order to amplify HEV of genotype 4.
